(19) United States
(12) Patent Application Publication
(10) Pub. No.: US 2003/0100107 A1
(43) Pub. Date: May 29, 2003

(54) Compositions and methods for generating differentiated human cells

(75) Inventor: Cesare Peschle, Rome (IT)

Correspondence Address: AKIN, GUMP, STRAUSS, HAUER & FELD, L.L.P., ONE COMMERCE SQUARE, 2005 MARKET STREET, SUITE 2200, PHILADELPHIA, PA, 19103, US

(73) Assignee: Thomas Jefferson University, Philadelphia, PA (US)

(21) Appl. No.: 10/007,574

(22) Filed: Nov. 9, 2001

Related U.S. Application Data
(63) Continuation-in-part of application No. 09/322,352, filed on May 28, 1999.
(60) Provisional application No. 60/087,153, filed on May 29, 1998.

(51) Int. Cl. C12N 5/08; G01N 33/567

(52) U.S. Cl. 435/372; 435/7.21

(57) ABSTRACT

The invention relates to methods of obtaining and expanding a purified population of long-term repopulating hematopoietic stem cells. The invention also relates to the uses of a purified population of long-term repopulating hematopoietic stem cells, for example by maintaining the stem cells in the presence of differentiated cells in order to induce the stem cells to differentiate to become cells of the same type.

CROSS-REFERENCE TO RELATED APPLICATIONS

This application is a continuation-in-part of U.S. patent application No. 09/322,352 which was filed on May 28, 1999 and claims priority, under 35 U.S.C. § 119(e), to U.S. Provisional Application No. 60/087,153, filed on May 29, 1998, both of which are hereby incorporated herein by reference in their entirety.

### STATEMENT REGARDING FEDERALLY SPONSORED RESEARCH OR DEVELOPMENT

Not applicable.

### REFERENCE TO A MICROFICHE APPENDIX

Not applicable.

### BACKGROUND OF THE INVENTION

Hematopoiesis in mammals is maintained by a pool of self-renewing hematopoietic stem cells (HSCs; Ogawa, 1993, Blood 81:2844-2853). HSCs feed into lineage(s)-committed undifferentiated hematopoietic progenitor cells (HPCs) with little or no self-renewal capacity (Ogawa, 1993, Blood 81:2844-2853). The HPCs in turn generate morphologically recognizable differentiated precursors and terminal cells circulating in peripheral blood.

Human HSCs are identified on the basis of their capacity for long-term hematopoietic repopulation in vitro and in vivo. Specifically, in vitro repopulation of an irradiated allogeneic stromal adherent layer in long term culture (LTC) of Dexter type has been observed. In Dexter type LTC, primitive HPCs and HSCs are assessed as five to eight week and twelve week LTC initiating cells (LTC-ICs), or cobblestone area forming cells (CAFCs; Sutherland et al., 1990, Proc. Natl. Acad. Sci. USA 87:3584-3588; Valtieri et al., 1994, Cancer Res. 54:4398-4404; Hao et al., 1996, Blood 88:3306-3313; Breems et al., 1996, Blood 87:5370-5378). Particularly, short term repopulating primitive HPCs have been identified in five to eight week LTC, whereas long-term repopulating putative HSCs have been identified in twelve week LTC (Sutherland et al., 1990, Proc. Natl. Acad. Sci. USA 87:3584-3588; Larochelle et al., 1996, Nature Med. 2:1329-1337; Hao et al., 1996, Blood 88:3306-3313). Moreover, in vivo repopulation of severe combined immunodeficiency (SCID) mice at two months or non-obese diabetic SCID (SCID-NOD) mice at one and a half months after irradiation and HSC injection has been observed (Nolta et al., 1994, Blood 83:3041-3047; Bock et al., 1995, J. Exp. Med. 182:2037-2043).

In murine embryonic life (day 7.5 of gestation), a close developmental association of the hematopoietic and endothelial lineages takes place in the yolk sack blood islands, leading to the hypothesis that the two lineages share a common ancestor referred to as the hemoangioblast (Flamme et al., 1992, Development 116:435-439; Risau et al., 1995, Ann. Rev. Cell. Dev. Biol. 11:73-91).

Vascular endothelial growth factor (VEGF) and one of its receptors, VEGFRII, termed Flk1 in mice and KDR in humans, play a key role in early hemoangiogenesis. In fact, Flk1⁻ knock-out mice are unable to form blood islands and blood vessels (Shalaby et al., 1995, Nature 376:62-66). Differentiated murine embryonic stem cells treated with VEGF and the ligand for c-kit receptor at the embryoid stage give rise to primitive blast cells which generate the various hematopoietic lineages, these data suggest a role for VEGF at the level of primitive HPCs in murine embryonic hematopoiesis (Kennedy et al., 1997, Nature 386:488-492; Kabrun et al., 1997, Development 124:2039-2048). There are no data concerning the effect of expression or the function of KDR in human embryonic/fetal HSCs.

In post-fetal life, the VEGF/KDR system plays an important role in the endothelial lineage. Indeed, KDR and CD34 antigens are expressed on progenitors of human adult endothelial cells (Ashara et al., 1997, Science 275:964-967). Again, there are no data concerning the effect of expression or the function of KDR in human post-fetal HSCs, particularly long-term repopulating HSCs. Most studies have focused on examination of the effect of VEGF on partially purified HPCs. The results of these studies suggest that VEGF exerts an enhancing or inhibitory effect on bone marrow (BM) HPC colony formation stimulated by diverse hematopoietic growth factors (HGFs) and a stimulatory effect on hematopoietic cells in normal mice (Broxmeyer et al., 1995, Int. J. Hematol. 62:203-215; Gabrilovich et al., 1998, Blood 92:4150-4166). In addition, KDR mRNA is expressed in cord blood (CB) and BM partially purified HPCs, while VEGF does not affect CB HPC colony formation but exerts an anti-apoptotic action on irradiated HPCs (Katoh et al., 1995, Cancer Res. 55:5687-5692).

There is a need in the art for efficient methods of purifying and characterizing long term repopulating HSCs and stem cells of other tissues and for methods of ex vivo expansion of these cells. In addition, there is a need in the art for methods of treating a variety of diseases using HSCs and other stem cells. The present invention satisfies these needs.

### BRIEF SUMMARY OF THE INVENTION

The invention relates to a method of generating a differentiated human cell of a selected type. The method comprises maintaining an isolated human KDR⁺ stem cell in the presence of a differentiated mammalian (e.g., human) cell of the selected type. In this environment, the stem cell differentiates to become a differentiated human cell of the selected type. The stem cell can be maintained in the presence of the differentiated mammalian cell by maintaining it in contact with the mammalian cell or by maintaining it separated from the mammalian cell by a porous barrier (i.e. a barrier that intermixing of stem and mammalian cells, but permits fluid communication between the media in which the stem and mammalian cells are suspended. Alternatively, the stem cells can be maintained in a medium conditioned to reflect the presence of the differentiated mammalian cell therein (e.g., a medium in which the mammalian cell had previously been maintained or a synthetic medium formulated to replicate small molecules normally released by the mammalian cell in culture). The stem cell can be maintained in the presence of the differentiated cell in vivo (e.g., at the site of a damaged tissue) or in vitro (e.g., in a synthetic or other culture medium in a commercially available cell culture apparatus).

KDR⁺ stem cells can be induced to differentiate into cells of ectodermal, mesodermal, or endodermal cell types. By way of example, a KDR+ stem cell can be induced to differentiate into a skeletal muscle cell, a myocardial cell, an epithelial cell, an endothelial cell, a cartilage cell, a retinal cell, a lens cell, a bone cell, a fat cell, a peripheral nerve cell, a differentiated hematopoietic cell, a marrow stromal cell, a hepatocyte, a splenocyte, a keratinocyte, a fibroblast, a lymphoid cell, or a central nervous system cell using the methods described herein.

KDR⁺ stem cells can be isolated from substantially any tissue that contains stem cells. For example, they can be isolated from a hematopoietic tissue of an embryonic, fetal, or post-natal (e.g., adult or juvenile) human. Examples of embryonic tissues from which KDR⁺ HSCs can be isolated include aorta-gonad-mesonephros region tissues, yolk sac, and liver. Examples of fetal tissues from which KDR⁺ HSCs can be isolated include liver, bone marrow, and peripheral blood. Examples of post-natal tissues from which KDR⁺ HSCs can be isolated include cord blood, bone marrow, normal and mobilized peripheral blood, liver, and spleen. KDR⁺ stem cells can be isolated from tissues in a variety of ways, such as by using a reagent (e.g., an antibody or a conjugated VEGF) that binds specifically with KDR.

The invention includes a method of repairing a damaged human tissue. The method comprises maintaining an isolated human KDR⁺ stem cell in the presence of a differentiated mammalian cell of a tissue (whether it is damaged or non-damaged) of the same type as the damaged tissue (or by maintaining the stem cell in a medium conditioned to reflect the presence therein of the differentiated cell). Following this treatment, the stem cell differentiates to become an altered cell that is differentiated from the initial stem cell. The altered cell can be a stem cell that has acquired the capacity to generate a tissue other than that of the tissue of its original residence (i.e., a tissue-exposed stem cell), even if the functionally different stem cell cannot be phenotypically distinguished until the stem cell further differentiates. The altered cell can also be a precursor or a terminally differentiated cell of the same type as the damaged tissue. The altered cell is provided to the damaged tissue, and the tissue is thereby repaired (i.e., either because altered cells of the damaged tissue type have been provided or because altered cells capable of differentiating to cells of the damaged tissue type have been provided). Damaged tissues that can be repaired in this manner include those associated with a disorders such as stroke, ischemia, myocardial infarction, coronary artery disease, spinal cord injury, age-related tissue damage, Alzheimer's disease, Parkinson's disease, liver fibrosis, liver cirrhosis, chronic obstructive pulmonary disorder, compartment syndrome, multiple sclerosis, chronic inflammation, chronic infection, macular degeneration, and cataracts.

The invention also includes a method of rejuvenating an age-damaged human tissue. The method comprises maintaining an isolated human KDR⁺ stem cell in the presence of a differentiated mammalian cell of a tissue (whether it is age-damaged or not) of the same type as the damaged tissue (or by maintaining the stem cell in a medium conditioned to reflect the presence therein of the differentiated cell). Following this treatment, the stem cell acquires the capacity to generate a tissue other than that of the tissue of its original residence . The altered cell can be a stem cell, a precursor of a cell of the same type as the age-damaged tissue, or a terminally differentiated cell of the same type as the age-damaged tissue. The altered cell is provided to the age-damaged tissue, and the tissue is thereby rejuvenated (i.e., either because altered cells of the damaged tissue type have been provided or because altered cells capable of differentiating to cells of the age-damaged tissue type have been provided).

The invention includes a method of obtaining a cell population enriched for long-term repopulating human hematopoietic stem cells (HSCs). The method comprises obtaining a population of cells from human hematopoietic tissue and isolating a population of KDR⁺ cells therefrom, thereby obtaining a cell population enriched for long-term repopulating human HSCs. In one aspect, the human hematopoietic tissue is selected from the group consisting of embryonic hematopoietic tissue, fetal hematopoietic tissue, and post-natal hematopoietic tissue. In another aspect, the human hematopoietic tissue is an embryonic hematopoietic tissue selected from the group consisting of aorta-gonad-mesonephros tissue, yolk sac, and embryonic liver. In yet another aspect, the human hematopoietic tissue is a fetal hematopoietic tissue selected from the group consisting of fetal liver, fetal bone marrow, and fetal peripheral blood. In a further aspect, the human hematopoietic tissue is a post-natal hematopoietic tissue selected from the group consisting of cord blood, bone marrow, normal peripheral blood, mobilized peripheral blood, hepatic hematopoietic tissue, and splenic hematopoietic tissue.

KDR⁺ cells can be isolated using a reagent which specifically binds KDR. In one aspect, the reagent is an antibody is selected from the group consisting of a polyclonal antibody and a monoclonal antibody (e.g., monoclonal antibody 260.4). KDR⁺ cells can be isolated using a conjugated VEGF, or a molecule derived therefrom.

The KDR⁺ stem cells used in the compositions and methods described herein can be starvation-resistant long-term repopulating human hematopoietic stem cells, such as an enriched population of long-term repopulating human hematopoietic stem cells obtained by obtaining a population of cells from human hematopoietic tissue and isolating a population of KDR⁺ cells therefrom.

In one aspect, a KDR⁺ cell comprises an isolated nucleic acid. The can be selected from the group consisting of a nucleic acid encoding adenosine deaminase, a nucleic acid encoding beta-globin, a nucleic acid encoding multiple drug resistance, an antisense nucleic acid complementary to a human immunodeficiency virus nucleic acid, an antisense nucleic acid complementary to a nucleic acid encoding a cell cycle gene, and an antisense nucleic acid complementary to a nucleic acid encoding an oncogene. The isolated nucleic acid can be operably linked to a promoter/regulatory sequence, such as one selected from the group consisting of a retroviral long terminal repeat and the cytomegalovirus immediate early promoter.

The invention includes a method of obtaining a purified population of long-term repopulating human HSCs. The method comprises obtaining a population of cells from human hematopoietic tissue, isolating a population of hematopoietic progenitor cells (HPCs) therefrom, and isolating a population of KDR⁺ cells from the population of HPCs, thereby obtaining a purified population of long-term repopulating human HSCs. The human hematopoietic tissue can be an embryonic hematopoietic tissue, a fetal hematopoietic tissue, or a post-natal hematopoietic tissue. For example, the hematopoietic tissue can be an embryonic tissue selected from the group consisting of a aorta-gonad-mesonephros region tissue, yolk sac, and liver, a fetal tissue selected from the group consisting of liver, bone marrow, and peripheral blood, or a post-natal tissue selected from the group consisting of cord blood, bone marrow, normal peripheral blood, mobilized peripheral blood, hepatic hematopoietic tissue, and splenic hematopoietic tissue.

The HPCs can be isolated using at least one method selected from the group consisting of isolation of cells expressing an early marker using antibodies specific for said marker, isolation of cells not expressing a late marker using antibodies specific for said late marker, isolation of cells based on a physical property of said cells, and isolation of cells based on a biochemical/biological property of said cells. Examples of early markers include CD34, Thy-1, c-kit receptor, flt3 receptor, AC133, VEGF receptor I, VEGF receptor III, Tie1, Tek, and basic fibroblast growth factor receptor. Examples of late markers include lineage (lin) markers.

HPCs can be obtained from a hematopoietic tissue using an antibody which specifically binds CD34 to select a population of CD34⁺ HPCs. KDR⁺ cells can be isolated from the CD34⁺ HPCs using an antibody which specifically binds KDR, such as monoclonal antibody 260.4.

HPCs can also obtained from a hematopoietic tissue using an antibody which specifically binds CD34 to select a population of CD34⁻ cells. HPCs can be obtained from the CD34⁻ cells using an antibody which specifically binds a lineage marker to select CD34⁻lin⁻ cells. KDR⁺ cells can be isolated from the CD34⁻lin⁻ cells, for example using an antibody which specifically binds KDR, such as monoclonal antibody is 260.4.

The invention also includes a method of expanding ex vivo human HSCs for use in in vivo therapy. The method comprises obtaining a population of KDR⁺ HSC according to the above-described method and incubating this cell population in the presence of VEGF, preferably associated with at least one other growth factor. As the data presented herein establish, treatment of the CD34⁺KDR⁺ cell population with VEGF and other HGFs results in a significant increase in the number of HSCs and/or HPCs. In one aspect, CD34⁺KDR⁺ cells are first seeded in starvation culture as described herein and then stimulated to grow in the culture supplemented with both VEGF and selected HGFs (including, for example, flt3 ligand or kit ligand), thus expanding exponentially (i.e., more than 10- to 100-fold) the number of primitive hematopoietic cells in the culture that exhibit HSC characteristics. In another aspect, addition of both VEGF and other suitable HGFs, as indicated herein, results in a marked amplification of the generated primitive HPCs, i.e., approximately a more than 100-fold amplification of CD34⁺CD38⁺ HPCs. The aforementioned HGFs include flt3 ligand, kit ligand, thrombopoietin, basic fibroblast growth factor, interleukin 6, interleukin 3, interleukin 11, granulomonocytic colony-stimulatory factor, granulocytic colony-stimulatory factor, monocytic colony-stimulatory factor, erythropoietin, angiopoietin, and hepatocyte growth factor.

The invention includes a blood substitute comprising progeny cells generated from an isolated purified population of long term repopulating human HSCs. The progeny cells can include one or more of red blood cells, neutrophilic granulocytes, eosinophilic granulocytes, basophilic granulocytes, monocytes, dendritic cells, platelets, B lymphocytes, T lymphocytes, natural killer cells, differentiated precursors of these cell types, and non-differentiated progenitors of these cell types.

The invention also includes a chimeric non-human mammal comprising at least one isolated or purified long-term repopulating human HSC. The HSC can be introduced into the mammal by transplantation or blastocyst injection, for example. Suitable mammals include mice, rats, dogs, donkeys, sheep, pigs, horses, cows, and non-human primates.

The invention includes a method of inhibiting rejection of a transplanted organ. The method comprises ablating the bone marrow of a transplant recipient and administering to the recipient a multi-lineage engrafting dose of an isolated and purified long-term repopulating human HSC obtained from the hematopoietic tissue of the donor of said organ, thereby inhibiting rejection of a transplanted organ.

The invention includes a method of transplanting an autologous human HSC in a human. The method comprises obtaining a population of cells from the hematopoietic tissue of a human and isolating a population of non-malignant HSCs therefrom, ablating the bone marrow of the human, and administering at least one isolated non-malignant HSC to the human, thereby transplanting an autologous human HSC in a human.

The invention also includes a method of isolating a KDR⁺ cell. The method comprises selecting a cell expressing an antigen co-expressed with KDR, thereby isolating a KDR⁺ cell. For example, the co-expressed antigen can be VEGF receptor I or a VEGF receptor III.

The invention includes a method of isolating a KDR⁺ stem cell giving rise to at least one of a muscle cell, a hepatic oval cell, a bone cell, a cartilage cell, a fat cell, a tendon cell, and a marrow stroma cell. The method comprises isolating a KDR⁺ stem cell from hematopoietic tissue, thereby isolating a KDR⁺ stem cell giving rise to at least one of a muscle cell, a hepatic oval cell, a bone cell, a cartilage cell, a fat cell, a tendon cell, and a marrow stroma cell.

The invention includes a method of monitoring the presence of KDR⁺ stem cells in a human hematopoietic tissue in a human receiving therapy. The method comprises obtaining a sample of hematopoietic tissue from the human before, during, and after the therapy, and measuring the number of KDR⁺ stem cells in the sample, thereby monitoring the presence of KDR⁺ stem cells in a human hematopoietic tissue obtained from a human receiving therapy.

Other features and advantages of the invention will be apparent from the following detailed description, and from the claims.

### BRIEF DESCRIPTION OF THE SEVERAL VIEWS OF THE DRAWINGS

FIG. 1, comprises FIGS. 1A through 1G. FIGS. 1A through 1E are a series of graphs depicting representative results on KDR expression and distribution of CD34⁺ cells, as assessed by flow cytometry. KDR expression was detected by flow cytometry on bone marrow (BM; FIG. 1A), normal peripheral blood (PB; FIG. 1B), mobilized peripheral blood (MPB; FIG. 1C), and cord blood (CB; FIG. 1D) in CD34⁺ cells. Cells gated on physical parameters were analyzed for specific and nonspecific (isotype-matched) antibody reactivity (greater than about 40,000 cells were analyzed). The vertical axis represents detection of phycoerythrin-(PE-)conjugated anti-KDR antibody (FIGS. 1A through 1E). The horizontal axis represents detection of peridinin chlorophyll protein (PerCP; FIGS. 1A and 1D) or fluorescein isothiocyanate (FITC; FIGS. 1B, 1C, and 1E) conjugated anti-CD34. The percentage of CD34⁺KDR⁺ cells is indicated by numbers on each figure. The graph in FIG. 1E depicts representative gates for analysis and sorting of KDR⁺ (KDR^{bright}), KDR^{+/±} (KDR^{dim}) and KDR⁻CD34⁺ cells. A CB experiment is shown. FIG. 1F is an image of a gel depicting the reverse transcriptase polymerase chain reaction (RT-PCR) analysis detecting the presence of KDR mRNA in CD34⁺KDR⁺ versus CD34⁺KDR⁻CB sorted cell populations. FIG. 1G, comprising FIGS. 1G-1 through 1G-14, is a series of graphs depicting representative results on the expression of KDR and relevant early hematopoietic antigens in electronically gated CD34⁺ cells. Electronically gated CD34⁺ cells from BM (FIGS. 1G-1 through 1G-5), PB (FIGS. 1G-6 through 1G-8), MPB (FIGS. 1G-9 through 1G-1), and CB (FIGS. 1G-12 through 1G-14) were analyzed for expression of KDR and several early hematopoietic antigens. In FIG. 1G-1, the vertical axis represents detection of PerCP-conjugated anti-CD34 antibody and the horizontal axis represents FSC-Height. In FIG. 1G-2, the vertical axis represents detection of control PE and the horizontal axis represents control FITC. In each of FIGS. 1G-3 through 1G-14, the vertical axis represents detection of PE-conjugated anti-KDR antibody. In FIGS. 1G-3, 1G-6, 1G-9, and 1G-12, the horizontal axis represents detection of FITC-conjugated anti-CD38 antibody. In FIGS. 1G-4, 1G-7, 1G-10, and 1G-13, the horizontal axis represents detection of FITC-conjugated anti-Thy 1 antibody. In FIGS. 1G-5, 1G-8, 1G-11, and 1G-14, the horizontal axis represents detection of FITC-conjugated anti-c-kit antibody.

FIG. 2 comprises FIGS. 2A through 2L. FIGS. 2A through 2C are a series of graphs depicting the in vitro HPC/HSC assays of CD34⁺KDR⁺ cells. FIGS. 2A and 2B depict the HPCs in PB CD34⁺ KDR^{+/±}(open bars) and CD34⁺ KDR⁻ (shaded bars) cells assayed in cultures supplemented with a restricted (FIG. 2A) or large (FIG. 2B) spectrum of hematopoietic growth factors (HGFs). FIG. 2C depicts primary (open bars) and secondary (shaded bars) HPP-CFC colonies in PB CD34⁺KDR⁺ and CD34⁺KDR⁻ cells. Mean± SEM from 4 independent experiments is disclosed. FIG. 2D is a graph depicting the PB CD34⁺ (open circles), CD34⁺KDR⁻ (shaded squares), and CD34⁺KDR^{+/±} (open squares) cell LTC; at 5, 8, and 12 weeks, supernatant and adherent cells were assayed for HPCs. FIGS. 2E and 2F depict BM (FIG. 2E) and CB (FIG. 2F) CD34⁺KDR⁻ (shaded squares) and CD34⁺KDR⁺ (open squares) cell LTC analyzed for CAFC-derived colonies at 6, 9, and 12 weeks. Mean±SEM from three experiments is disclosed. FIGS. 2G through 2J are a series of graphs depicting LDA of 12 week LTC-ICs/CAFCs in CD34⁺KDR⁺ cells. FIG. 2G depicts LTC-IC frequency in PB CD34⁺, CD34⁺KDR^{+/±}, CD34⁺KDR⁺, and CD34⁺KDR⁻ cells. The mean±SEM for five separate VEGF⁺(shaded bars) or three separate VEGF⁻(open bars) experiments is shown. FIG. 2H depicts representative LDAs for PB CD34⁺ and CD34⁺KDR⁺ cells (100 replicates for the lowest cell concentration (e.g., 1 KDR⁺ cell) and decreasing replicate numbers for increasing cell concentrations, i.e., 50, 20, 10 wells with 2, 5, 10 KDR⁺ cells, respectively. FIGS. 2I and 2J depict CAFC frequency in KDR⁺ and KDR⁻ cells from BM (FIG. 2I) or CB (FIG. 2J). The mean±SEM for three separate experiments is shown. In FIGS. 2G, 2I, and 2J, the symbol ** indicates that p<0.01 when compared to the VEGF-group. In FIG. 2G, the symbol °° indicates that p<0.01 when compared to the other groups. FIG. 2K is a graph depicting the starvation of PB CD34⁺KDR^{+/±} or CD34⁺ KDR⁻ cells in single cell FCS⁻ free liquid phase culture supplemented with VEGF (shaded bars) or not supplemented with VEGF (open bars); the percentage of cells that survived at day 21 (mean±SEM from 3 separate experiments) is shown. FIG. 2L, comprising FIGS. 2L-1 and 2L-2, depicts the minibulk (2×10³ cells per milliliter) PB CD34⁺KDR^{+/±} starvation culture supplemented with VEGF; the limiting dilution assay (LDA) of LTC-IC frequency in the approximately 25% cells surviving on day 5 (FIG. 2L-1) and 25 (FIG. 2L-2) is shown.

FIG. 3 comprises FIGS. 3A-1 through 3H. FIGS. 3A-1 through 3C-7 are a series of graphs depicting representative results on the engraftment of BM CD34⁺KDR⁺ cells in NOD-SCID mice demonstrating the repopulating activity of 100 (FIG. 3A-4), 400 (FIG. 3A-3), 800 (FIG. 3A-2), and 1,600 (FIG. 3A-1) CD34⁺KDR⁺ cells in recipient mice. The positive and negative controls received CD34⁺ (FIG. 3A-5) and CD34⁺KDR⁻ (FIG. 3A-6) cells, respectively (FIG. 3A). FIGS. 3A and 3B depict human CD34⁺/CD45⁺ cell engraftment (FIG. 3A) and CD45⁺ cell dose-response (mean± SEM, three mice per group, r=0.99; FIG. 3B). In FIGS. 3A-1 through 3A-6, the vertical axis represents detection of PE-conjugated anti-CD34 antibody and the horizontal axis represents detection of FITC-conjugated anti-CD45 antibody. Dose-dependent engraftment was also observed in recipient PB and spleen. FIG. 3C, comprising FIGS. 3C-1 through 3C-7, depicts the expression of human hematolymphopoietic markers in a representative mouse injected with 1,600 CD34⁺KDR⁺ cells. In FIG. 3C-1, the vertical axis represents detection of PE-conjugated anti-CD33 antibody and the horizontal axis represents detection of FITC-conjugated anti-CD15 antibody. In FIG. 3C-2, the vertical axis represents detection of PE-conjugated anti-CD14 antibody and the horizontal axis represents detection of FITC-conjugated anti-CD45 antibody. In FIG. 3C-3, the vertical axis represents detection of PE-conjugated anti-CD71 antibody and the horizontal axis represents detection of FITC-conjugated anti-GPA antibody. In FIG. 3C-4, the vertical axis represents detection of PE-conjugated anti-CD41 antibody and the horizontal axis represents detection of FITC-conjugated anti-CD45 antibody. In FIG. 3C-5, the vertical axis represents detection of PE-conjugated anti-CD20 antibody and the horizontal axis represents detection of FITC-conjugated anti-CD19 antibody. In FIG. 3C-6, the vertical axis represents detection of PE-conjugated anti-CD4/CD8 antibody and the horizontal axis represents detection of FITC-conjugated anti-CD3 antibody. In FIG. 3C-7, the vertical axis represents detection of PE-conjugated anti-CD56 antibody and the horizontal axis represents detection of FITC-conjugated anti-CD 16 antibody.

FIGS. 3D through 3F are a series of graphs depicting representative results on the engraftment of BM CD34⁺KDR⁺ cells in NOD-SCID mice demonstrating the LDA of repopulating HSC frequency in CD34⁺KDR⁺ cells. Graded numbers of BM CD34⁺KDR⁺ cells were injected into recipient mice. The positive and negative controls received CD34⁺ and CD34⁺KDR⁻ cells, respectively. FIGS. 3D and 3E depict human CD45⁺ cells in BM of mice injected with 250, 50, 10 or 5 cells (3, 9, 6 and 6 mice per group, respectively; mean±SEM). FIG. 3F depicts human HPCs in BM of the 4 engrafted mice injected with 5 cells (mean± SEM) and the LDA according to single hit Poisson statistics. The bottom right panel depicts the PCR analysis of human alpha-satellite DNA (867 base pair band) in all scored colonies from a representative mouse that received 5 cells. The contents of the lanes are indicated in the figure, in addition lane 13 depicts a human DNA positive control, lane 14 depicts a no DNA template negative control, lane 15 comprises DNA from BM mononuclear cells of a non-transplanted mouse, and M.W. indicates a lane comprising molecular weight markers.

FIG. 3H, comprising FIGS. 3H-1 through 3H-8, is a series of graphs depicting the expression of informative human hematolymphopoietic markers in a representative mouse receiving 6,000 CB CD34⁺KDR⁺ cells as described herein. In FIG. 3H-1, the vertical axis represents detection of PE-conjugated anti-CD34 antibody and the horizontal axis represents FITC-conjugated anti-CD45 antibody. In FIG. 3H-2, the vertical axis represents detection of PE-conjugated anti-CD71 antibody and the horizontal axis represents FITC-conjugated anti-GPA antibody. In FIG. 3H-3, the vertical axis represents detection of PE-conjugated anti-CD33 antibody and the horizontal axis represents FITC-conjugated anti-CD15 antibody. In FIG. 3H-4, the vertical axis represents detection of PE-conjugated anti-CD14 antibody and the horizontal axis represents FITC-conjugated anti-CD45 antibody. In FIG. 3H-5, the vertical axis represents detection of PE-conjugated anti-CD41 antibody and the horizontal axis represents FITC-conjugated anti-CD45 antibody. In FIG. 3H-6, the vertical axis represents detection of PE-conjugated anti-CD20 antibody and the horizontal axis represents FITC-conjugated anti-CD19 antibody. In FIG. 3H-7, the vertical axis represents detection of PE-conjugated anti-CD4 antibody and the horizontal axis represents FITC-conjugated anti-CD3 antibody. In FIG. 3H-8, the vertical axis represents detection of PE-conjugated anti-CD56 antibody and the horizontal axis represents FITC-conjugated anti-CD16 antibody.

FIG. 4, comprising FIGS. 4A through 4C, is a series of graphs depicting representative results on the engraftment of BM CD34⁺KDR⁺ cells in primary (FIGS. 4A and 4B) and secondary (FIG. 4C) fetal sheep. The total estimated number of human CD34⁺, CD45⁺, glycophorin A⁺ (GPA⁺), and CD7⁺ cells generated in primary fetal sheep recipients transplanted with 3×10³ CD34⁺KDR^{+/±} (FIG. 4B) or 2.4×10⁵ CD34⁺KDR⁻ cells per fetus (FIG. 4 A; mean± SEM). The percentage of human CD45⁺ cells and total HPCs in BM of secondary sheep fetuses is depicted in FIG. 4C (mean± SEM).

### DETAILED DESCRIPTION OF THE INVENTION

The invention is based on the discovery that vascular endothelial growth factor receptor II (VEGFRII; KDR) is a key functional marker for long-term repopulating human HSCs. The identification of HSCs expressing KDR (i.e., KDR⁺ HSCs) serves to facilitate the development of improved methodology for the purification and characterization of long-term repopulating HSCs. The identification of KDR⁺ HSCs also serves to facilitate ex vivo expansion of purified HSCs by incubation of cells from hematopoietic tissue with VEGF combined with other hematopoietic growth factors (HGFs). Generation of chimeric animals (at the somatic level) through human HSC injection into an animal (preferably a mammalian) blastocyst generates human hematopoietic cells in this animal in vivo.

More broadly, it has been discovered that KDR is expressed on sub-populations of hematopoietic and non-hematopoietic cells that are enriched for primitive stem cells (e.g., on a subset of primitive cells in neurospheres, which represent a subset of neural stem cells). The KDR⁺ cell subset is endowed with stem cell activity, i.e., capacity for extensive self-renewal and wide spectrum differentiation, including differentiation in tissue other than the tissue in which the KDR⁺ cell normally occurs or from which it is isolated. Expression of KDR, therefore, is an indication that the KDR⁺ stem cells (i.e., those derived from any tissue, not just hematopoietic tissues) are in a highly non-differentiated state and retain the ability to differentiate into cells of numerous tissue types. Expression of KDR by stem cells is also an indication that the stem cells retain the ability to self-renew in their non-differentiated state (i.e., reproduce without becoming more highly differentiated). Thus, KDR⁺ stem cells isolated from substantially any tissue (e.g., KDR⁺ stem cells obtained from a hematopoietic, mesenchymal, muscle, or neural tissue) can be used in the compositions and methods described herein. Numerous methods are known for isolating stem cells from various tissues, and additional stem cell isolation techniques will, no doubt, continue to be discovered. Any of these methods can be combined with selection of KDR⁺ cells from an isolated stem cell population in order to isolate highly non-differentiated stem cells.

In another important aspect of the invention, it has been discovered that KDR⁺ stem cells can be induced to differentiate into a selected cell type by maintaining the KDR⁺ stem cells in the presence of differentiated cells of that type or in cell culture medium that has been conditioned to reflect the presence of differentiated cells of that type therein. Without being bound by any particular theory of operation, it is believed that cytokines or other small molecules secreted by differentiated cells (especially by damaged differentiated cells), interactions between the differentiated cells, interactions between differentiated cells and their extracellular matrix, or some combination of these factors are able to direct differentiation of KDR⁺ stem cells such that the stem cells become differentiated cells of the same type. Thus, for example, KDR⁺ stem cells that are isolated from a hematopoietic or neural tissue and that are maintained in the presence of muscle cells will differentiate to become muscle cells or myogenic progenitor cells. Similarly, KDR⁺ stem cells that are maintained in the presence of a culture medium containing molecules that are present in culture medium in which muscle cells are maintained will differentiate to become muscle cells or myogenic progenitor cells.

These observations indicate that KDR⁺ stem cells can be induced to differentiate into a selected cell type in a variety of ways. For example, the stem cells can be maintained (in vitro or in vivo) in the presence of differentiated cells of the selected type in such a way that the stem cells can contact (or must contact) the differentiated cells. Alternatively, the stem cells can be maintained in the same medium as differentiated cells of the selected type, but in an arrangement such that the stem cells and differentiated cells are not able to contact one another (e.g., the two cell types are separated by a porous barrier, such as an ultrafiltration membrane, through which small proteins (e.g., MW<50,000) can pass, but through which cells cannot pass). As still another alternative, a culture medium that is conditioned to reflect the presence of the selected differentiated cell type therein can be made, and the KDR⁺ stem cells can be cultured in that conditioned medium so that they differentiate to become cells of the selected type. Such conditioned medium can be made by actually culturing differentiated cells of the selected type therein, and preferably by culturing damaged, differentiated cells of the selected type therein. The cells can be removed (e.g., by filtration, centrifugation, or starvation) prior to culturing the stem cells in the medium. The conditioned medium can also be prepared by adding molecules (e.g., cytokines, proteins, or other small molecules) that are normally produced by a culture of differentiated cells of the selected type to a culture medium.

At a clinical level, purified KDR⁺ HSCs (or other KDR⁺ stem cells) serve as key innovative tools for allogeneic or autologous HSC transplantation, as applied in leukemia/lymphoma, solid tumors, hematopoietic diseases and autoimmune disorders, and for HSC-based gene therapy for treatment of a large spectrum of hereditary of acquired disorders affecting hematopoiesis and/or lymphopoiesis (e.g., AIDS). In addition, following in vitro expansion and differentiation of purified KDR⁺ HSCs, the KDR⁺ HSC progeny, for example, red blood cells, granulocytes and/or platelets, are useful in transfusion medicine. Isolated, expanded, or isolated and expanded KDR⁺ stem cells can be transplanted into a mammal such as a human to replace, rejuvenate, or supplement a damaged or excised tissue. Preferably, the stem cells are removed from a tissue of the intended recipient, isolated or expanded ex vivo, and then returned to the same recipient. However, KDR⁺ stem cells can be removed from one human donor and transplanted into a different human recipient, particularly if the donor and recipient are closely related by heredity. Thus, for example, KDR⁺ stem cells isolated from cord blood of an individual can be expanded, differentiated, or both in vitro prior to implantation (e.g., local or systemic injection) in a sibling of the individual in order to replace, rejuvenate, or supplement a tissue of the sibling.

The invention thus includes a method of obtaining a cell population enriched for long-term repopulating human HSCs. The method comprises obtaining a population of cells from human hematopoietic tissue. From the cells obtained from the hematopoietic tissue, cells expressing KDR on their surface (i.e., KDR⁺ cells) are isolated. In one embodiment, the KDR⁺ cells are isolated using monoclonal antibody 260.4 (KDR2). However, the present invention is not limited to isolation of KDR⁺ cells using any particular antibody. Rather, the present invention encompasses using any antibody (e.g., a polyclonal antibody) which specifically binds KDR to isolate KDR⁺ cells. The invention includes a population of KDR⁺ cells obtained using any of the methods described herein.

The invention also includes a method of obtaining a cell population enriched for long-term repopulating human HSCs wherein KDR⁺ cells are isolated using a conjugated VEGF. This method relies on the affinity of the KDR-VEGF receptor-ligand interaction to select cells expressing KDR on their surfaces by binding such cells, via the KDR present on the surface of the cell, to VEGF conjugated to, for example, a solid support matrix (e.g., an agarose or other surface, such as agarose beads). Thus, the VEGF-conjugate can be used to affinity-purify the KDR expressing cells by standard methods known in the art.

The KDR⁺ cell fraction isolated with any of the methods described herein will likely not be comprised solely of long-term repopulating HSCs; instead, the fraction can include other cells such as megakaryocytes, endothelial cells, and the like, which express KDR but which are not HSCs. These cells can be, and preferably are, removed from the KDR⁺ HSCs by various methods known in the art, based on the physical, biochemical, immunological, and/or morphological differences between these cells and KDR⁺ undifferentiated hematopoietic progenitors and KDR⁺ stem cells. However, for purposes of the present invention, non-HSC KDR⁺ cells need not be removed from the KDR⁺ fraction isolated from human hematopoietic tissue or from another KDR⁺ stem cell-containing tissue.

Human hematopoietic tissues include embryonic, fetal, and post-natal hematopoietic tissues. Embryonic hematopoietic tissues include, for example, aorta-gonad-mesonephros region tissues, yolk sac, and liver. Fetal hematopoietic tissues include liver, bone marrow, and peripheral blood. Post-natal hematopoietic tissues, in turn, include cord blood, bone marrow, hepatic hematopoietic tissue, splenic hematopoietic tissue, normal peripheral blood, and mobilized peripheral blood.

The invention also includes a method of obtaining an enriched population of long-term repopulating HSCs (or other KDR⁺ stem cells) that is starvation-resistant. Starvation-resistant cells are obtained by growing the KDR⁺ cells in mini-bulk culture under starvation conditions as described herein. Starvation-resistant cells obtained following culture constitute much fewer cells than are originally placed in serum-free culture in the absence of any HGF treatment, except for VEGF addition. However, the resulting starvation-resistant cells comprise a much higher percentage of putative HSCs than an otherwise identical population of cells that are not grown under identical conditions, therefore, putative HSCs are further enriched in the KDR⁺ fraction as a result of the starvation selection. The particular conditions for starvation culture are set forth herein. One skilled in the art, based upon the disclosure provided herein, would appreciate that the particular conditions, e.g., the precise number of days, can be varied so long as serum and HGFs are not added into the medium in any significant amount. The resultant starvation-resistant cells, which are enriched for in vitro long-term repopulating HSCs, can then be used in a wide variety of applications as described herein. The invention includes a cell obtained by the above-disclosed method of obtaining a cell population enriched for long-term repopulating human HSCs.

Further, the invention includes a KDR stem or progenitor cell obtained using any of the methods described herein, wherein the cell comprises an isolated nucleic acid. The nucleic acid can be introduced into the cell using any method for introducing a nucleic acid into a cell; such methods are known in the art and are described, for example, in Sambrook et al. (1989, In: Molecular Cloning: A Laboratory Manual, Cold Spring Harbor Laboratory Press, New York), and Ausubel et al. (1997, In: Current Protocols in Molecular Biology, Green & Wiley, New York). These methods include calcium phosphate precipitation transfection, DEAE dextran transfection, electroporation, microinjection, liposome-mediated transfer, chemical-mediated transfer, ligand-mediated transfer, and recombinant viral vector transfer, and the like.

The nucleic acid which can be transfected and/or transduced into the cell includes a nucleic acid such as that encoding adenosine deaminase, beta-globin, and multi-drug resistance. Thus, the cell can, if the nucleic acid is expressed, be used to provide the protein encoded thereby to the cell and/or to the extracellular milieu. The present invention is not limited to these particular nucleic acids. Instead, a wide variety of nucleic acids encoding any known protein can be transfected into the cell of the invention. Thus, the invention includes nucleic acid products which are useful for the treatment of various disease states in a mammal. Such nucleic acids and associated disease states include: DNA encoding glucose-6-phosphatase, associated with glycogen storage deficiency type 1A; DNA encoding phosphoenolpyruvate-carboxykinase, which is associated with Pepck deficiency; DNA encoding galactose-1 phosphate uridyl transferase, which is associated with galactosemia; DNA encoding phenylalanine hydroxylase, which is associated with phenylketonuria; DNA encoding branched chain alpha-ketoacid dehydrogenase, which is associated with Maple syrup urine disease; DNA encoding fumarylacetoacetate hydrolase, which is associated with tyrosinemia type 1; DNA encoding methylmalonyl-CoA mutase, which is associated with methylmalonic acidemia; DNA encoding medium chain acyl CoA dehydrogenase, which is associated with medium chain acetyl CoA deficiency; DNA encoding omithine transcarbamylase, which is associated with omithine transcarbamylase deficiency ; DNA encoding argininosuccinic acid synthetase, which is associated with citrullinemia; DNA encoding low density lipoprotein receptor protein, which is associated with familial hypercholesterolemia; DNA encoding UDP-glucouronosyltransferase, which is associated with Crigler-Najjar disease; DNA encoding adenosine deaminase, which is associated with severe combined immunodeficiency disease; DNA encoding hypoxanthine guanine phosphoribosyl transferase, which is associated with Gout and Lesch-Nyan syndrome; DNA encoding biotinidase, which is associated with biotinidase deficiency; DNA encoding beta-glucocerebrosidase, which is associated with Gaucher disease; DNA encoding beta-glucuronidase, which is associated with Sly syndrome; DNA encoding peroxisome membrane protein 70 kilodaltons, which is associated with Zellweger syndrome; DNA encoding porphobilinogen deaminase, which is associated with acute intermittent porphyria; DNA encoding alpha-1antitrypsin for treatment of alpha-1 antitrypsin deficiency (emphysema); DNA encoding erythropoietin for treatment of anemia due to thalassemia or to renal failure; and, DNA encoding insulin for treatment of diabetes. Such DNAs and their associated diseases are reviewed in Kay et al. (1994, T.I.G. 10:253-257) and in Parker and Ponder (1996, “Gene Therapy for Blood Protein Deficiencies,” In: Gene Transfer in Cardiovascular Biology: Experimental Approaches and Therapeutic Implications, Keith and March, eds.).

A human long-term repopulating HSC (or other KDR⁺ stem cell) which is able to engraft a recipient and which comprises a nucleic acid is useful for gene therapy. That is, such a stem cell can, when introduced into an animal, express the nucleic acid, thereby producing the encoded protein and correcting a genetic defect in a cell. The nucleic acid can encode a protein which is not otherwise present in sufficient and/or functional quantity, such that providing the nucleic acid corrects a genetic defect in the cell. The nucleic acid can encode a protein which is useful as a therapeutic agent in the treatment or prevention of a particular disease condition or disorder or of symptoms associated therewith. Thus, long-term repopulating human HSCs are useful therapeutics, allowing the expression of an isolated nucleic acid present in such cell.

The invention also includes a cell transfected with an antisense nucleic acid that is complementary to a nucleic acid encoding a retrovirus (such as human immunodeficiency virus), a cell cycle gene, or an oncogene. Under certain circumstances, it is useful to inhibit expression of a nucleic acid. Certain molecules, including antisense nucleic acids and ribozymes, are useful in inhibiting expression of a nucleic acid complementary thereto.

Antisense molecules and their use for inhibiting gene expression are known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined herein, to at least a portion of a specific mRNA molecule (Weintraub, 1990, Scientific American 262:40). In the cell, antisense nucleic acids hybridize to the corresponding mRNA, forming a double-stranded molecule thereby inhibiting transcription, translation, or both, of genes.

The use of antisense methods to inhibit the translation of genes is known in the art, and is described, for example, in Marcus-Sakura (1988, Anal. Biochem. 172:289). Such antisense molecules can be provided to the cell via genetic expression using DNA encoding the antisense molecule as taught by Inoue, 1993, U.S. Pat. No. 5,190,931.

Alternatively, antisense molecules of the invention can be made synthetically and then provided to the cell. Antisense oligomers of about 15 nucleotides are preferred, since they are easily synthesized and introduced into a target cell. Synthetic antisense molecules contemplated by the invention include oligonucleotide derivatives known in the art which have improved biological activity compared to unmodified oligonucleotides (see Cohen, supra; Tullis, 1991, U.S. Pat. No. 5,023,243).

Ribozymes are another nucleic acid that can be transfected into the cell to inhibit nucleic acid expression in the cell. Ribozymes and their use for inhibiting gene expression are also known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053). Ribozymes are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). A major advantage of this approach is that, because they are sequence-specific, only mRNAs with particular sequences are inactivated.

There are two basic types of ribozymes, namely, tetrahymena-type and hammerhead-type (Hasselhoff, 1988, Nature 334:585). Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules.

Ribozymes useful for inhibiting the expression of the proteins of interest can be designed by incorporating target sequences into the basic ribozyme structure which are complementary to the mRNA sequence of the nucleic acid encoding the protein of interest. Ribozymes targeting an immunodeficiency virus nucleic acid, a cell cycle gene, and an oncogene can be synthesized using commercially available reagents (Applied Biosystems, Inc., Foster City, Calif.) or they can be expressed from DNA encoding them.

The invention includes a cell comprising an isolated nucleic acid, wherein the nucleic acid is operably linked to a promoter/regulatory sequence. Accordingly, expression of the nucleic acid in cells which do not normally express the nucleic acid can be accomplished by transfecting the cell with a nucleic acid operably linked to a promoter/regulatory sequence which serves to drive expression of the nucleic acid. Many promoter/regulatory sequences useful for driving constitutive expression of a gene are available in the art and include the cytomegalovirus immediate early promoter enhancer sequence, the SV40 early promoter, the Rous sarcoma virus promoter, and the like. Inducible and tissue specific expression of the nucleic acid operably linked thereto can be accomplished by placing the nucleic acid under the control of an inducible or tissue specific promoter/regulatory sequence. Examples of tissue specific or inducible promoter/regulatory sequences which are useful for this purpose include the MMTV long terminal repeat (LTR) inducible promoter and the SV40 late enhancer/promoter. In addition, promoters which are known in the art which are induced in response to inducing agents such as metals, glucocorticoids, and the like, are also contemplated in the invention. Thus, it will be appreciated that the invention includes the use of any promoter/regulator sequence which is either known or is heretofore unknown, which is capable of driving expression of the nucleic acid operably linked thereto.

The invention also includes a method of obtaining a purified population of human HSCs (or other KDR⁺ stem cells). The method comprises two steps. The first step involves purification of HPCs from cells obtained from one or more human hematopoietic tissues (or of another type of progenitor cell from another tissue). Such progenitor cells, or blasts, can be purified by various methods capitalizing on the difference(s) in a physical property (e.g., the cell density), a biochemical/biological property (e.g., the ability to take up a dye), and/or the expression of various surface markers, using established procedures known in the art.

In one embodiment, CD34⁺ HPCs were isolated using established procedures described herein, wherein the CD34⁺ HPCs are obtained from embryonic fetal liver (FL), cord blood (CB), adult bone marrow (BM), normal peripheral blood (PB), and mobilized peripheral blood (MPB). The preferred method for purification of these cells is by use of the MiniMACS™ Multisort CD34 isolation system (Miltenyi, Bergisch Gladbach, Germany). However, other methods known in the art for purification of HPCs, including CD34⁺ cells, or methods to be developed, can also be used to practice the present invention. Although the CD34 marker was used to isolate HPCs, other early markers such as c-kit, CD38, Thy-1, and AC133, and the like, can also be used to isolate such cells.

In addition, CD34⁻ cells which are also lin⁻ can also be used as the population of hematopoietic cells which are then processed according to the second step of the method. As disclosed in the examples, CD34⁻lin⁻KDR⁺ cells also comprise HSCs, and these cells are able to engraft non-human animals just as CD34⁺KDR⁺ cells also engraft these animals. Thus, the CD34⁻lin⁻ cells also comprise a useful population enriched for undifferentiated cells from which long-term repopulating human hematopoietic cells can be isolated.

CD34⁺ versus CD34⁻ cells and lin⁺ versus lin⁻ cells can be separated from each other by, for example, fluorescence activated cell sorting (FACS) as disclosed herein. However, the present invention is not limited to this method of selecting cells on the basis of the expression of various cell surface markers. Rather, other methods known in the art for obtaining fractions of cell populations are also encompassed by the present invention.

In the second step, the human HPCs (or other hematopoietic cells) isolated previously are selected for expression of KDR. In one embodiment, the HPCs were separated by cell sorting into CD34⁺KDR⁺ (KDR^{bright}), CD34⁺KDR^{+/±}(KDR^{dim}) and CD34⁺KDR⁻ cells using anti-KDR monoclonal antibody (i.e., the 260.4 clone available from Gesellschaft fur Biologische Forschung, GBF, Braunschweig, Germany, or any other monoclonal antibody (MoAb) or molecule recognizing KDR⁺ cells, such as KDR2 antibody (MoAb 260.4) available from Sigma Chemical Company, St. Louis, Mo.).

Other methods known in the art for separation of cell subsets, or methods to be developed, can also be used to practice the present invention. The methods described herein for purification of KDR⁺ cells can be modified by using any other reagent or combination of reagents such as any MoAb or combination of MoAbs used together with any reagent (e.g., any combination of monoclonal and polyclonal antibodies) which specifically bind KDR. Thus, the present invention is not limited to using MoAb 260.4, or any other antibody, to isolate cells expressing KDR.

Other early markers besides CD34 can be used to select human long-term repopulating HSCs in conjunction with KDR. As an example, AC133 is expressed on immature HPCs and stem cells (Miraglia et al., 1997, Blood 90:5013-5021; Yin et al., 1997, Blood 90:5002-5012). AC133 MoAbs recognize 20-60% of CD34⁺ cells including CD34^{+bright}, CD38^{-/dim}, HLA⁻DR⁻, CD90⁺, and CD117⁺ cells. Thus, instead of using CD34⁺ or CD34⁻ cells expressing KDR, AC133⁺ or AC133⁻ cells expressing KDR can be utilized. The invention includes all reagents when used together with any reagent recognizing KDR, such as other early markers including c-kit receptor, Thy-1, VEGF receptor I, VEGF receptor III, Tie1, Tek, basic fibroblast growth factor receptor, flt3 receptor, and AC133, as well as the selection of cells which are negative for late markers such as lin, and the like.

Receptor-type tyrosine kinases (RTKs) constitute a family of proteins involved in growth and developmental processes activating various cellular responses during embryogenesis and adult life. To further characterize CD34⁺ that express KDR, RT-PCR for detection of various RTKs (VEGFRI (fltl), VEGFRIII (flt1), Tie1, and Tek) in these KDR⁺ subsets was applied by using a RT-PCR methodology described in Ziegler et al. (1999, Blood 93:3355-3368). RT-PCR analysis provided evidence that RTKs such as Flt1, Flt4, Tie1, and Tek, were expressed at the transcriptional level in small numbers of highly purified CD34⁺KDR⁺ cells. Thus, CD34⁺ cells expressing KDR can be further subdivided into subsets that express RTKs or do not express RTKs, by using RTK specific antibodies or any other reagent recognizing RTKs. The invention thus includes all technologies/methodologies aimed to further subdivide the CD34⁺ population that are KDR⁺ by means of reagents recognizing the above mentioned RTKs, any other RTKs, or any other cell surface structure expressed on KDR⁺ populations.

Thus, the invention includes a method of isolating a KDR⁺ cell by selecting cells expressing an antigen co-expressed with KDR on the surface of cells. Antigens co-expressed with KDR include, for example, VEGFRI (flt1) and VEGFRIII (flt4). Thus, KDR⁺ cells can be isolated by selecting for cells that express VEGFRI and/or VEGFRIII which are known to co-express with KDR.

The purified human HSCs in the KDR⁺ and KDR⁻ cell population are assayed based upon their capacity for long term hematopoietic repopulation in vitro or in vivo. In parallel, the HPCs present in these two cell populations are assayed for their capacity for in vitro short term generation of a hematopoietic progeny. The long-term repopulation HSCs, defined according to the criteria described in the Examples section, are virtually exclusively contained within the CD34⁺KDR⁺ and CD34⁺KDR^{+/±} fractions. Conversely, unilineage and bilineage HPCs are almost exclusively contained within the CD34⁺KDR⁻ fraction. This method of purification of HSCs from CB, adult BM, and PB or MPB, yields a suitable number of HSCs for in vitro and in vivo clinical use. The most preferred sources of purified HSCs are post-natal hematopoietic tissues (e.g., CB, adult BM, PB, and MPB). However, other hematopoietic tissue sources include, for example, embryonic hematopoietic tissue (e.g., aorta-gonad-mesonephros region tissue, yolk sac and embryonic liver), fetal hematopoietic tissue (e.g., fetal liver, fetal bone marrow, and fetal peripheral blood).

The long-term repopulating HSCs can be further purified by growing CD34⁺KDR⁺ or CD34⁺KDR^{+/±} cells in mini-bulk culture under starvation conditions, as described herein. Starvation-resistant cells obtained following culture constitute approximately 10-25% of the initial number of peripheral blood cells, or approximately 30-70% of the initial number of CB cells, placed in serum-free culture in the absence of any HGF treatment, except for VEGF addition. However, the resulting starvation-resistant cells are essentially 100% KDR⁺ and comprise approximately≧80-95% putative HSCs, thereby being greatly enriched for HSCs as a result of the starvation selection. The particular conditions for starvation culture are set forth herein. The particular conditions, e.g., the precise number of days, can be varied so long as serum and HGFs are not added into the medium in any significant amount. The resultant starvation-resistant cells, which are greatly enriched for in vitro long-term repopulating HSCs, can be used in a wide variety of applications, as described herein. The invention includes cells isolated by this method.

The invention includes a method of expanding ex vivo human HSCs for use in in vivo therapy. The method comprises obtaining a population of KDR⁺ HSC according to the above-described method and incubating this cell population in the presence of VEGF, preferably associated with at least one other growth factor. As data herein establish, treatment of the CD34⁺KDR⁺ cell population with VEGF and other HGFs results in a significant increase in the number of HSCs and/or HPCs. In one aspect, CD34⁺KDR⁺ cells are first seeded in starvation culture as described herein and then stimulated to grow in the culture supplemented with both VEGF and selected HGFs (including, for example, flt3 ligand or kit ligand), thus expanding exponentially (i.e., more than 10- to 100-fold) the number of primitive hematopoietic cells in the culture that exhibit HSC characteristics. In another aspect, addition of both VEGF and other HGFs results in a marked amplification of the generated primitive HPCs, i.e., approximately a more than 100-fold amplification of CD34⁺CD38⁺ HPCs. The aforementioned HGFs include flt3 ligand, kit ligand, thrombopoietin, basic fibroblast growth factor, interleukin 6, interleukin 3, interleukin 11, granulomonocytic colony-stimulatory factor, granulocytic colony-stimulatory factor, monocytic colony-stimulatory factor, erythropoietin, angiopoietin, and hepatocyte growth factor. Non-hematopoietic KDR⁺ stem cells can be expanded by similar methods, including using tissue-specific growth factors.

Purified HPCs can be differentiated for use in transfusion medicine. In this regard, a combined step procedure is applied to cells in culture. In one step, the purified CD34⁺KDR⁺ and/or the CD34⁻lin⁻KDR⁺ population of long-term repopulating human HSCs is amplified, which results in the generation of HSCs/HPCs by addition of VEGF and other suitable HGFs as described herein. In another step, the generated HSC/HPC population is grown in culture conditions which selectively induce the HPCs to differentiate and mature through one of the erythroid, megakaryocytopoietic, granulopoietic/neutrophilic, monocytopoietic pathway, and other hematopoietic pathways, yielding granulopoietic/eosinophilic, basophilic, or dendritic cells, B or T lymphopoietic, or NK cell pathways (Labbaye et al., 1995, J. Clin. Invest. 95:2346-2358; Guerriero et al., 1995, Blood 86:3725-3736; Gabbianelli et al., 1995, Blood 86:1661-1670). Other methods known in the art for hematopoietic cell production, or methods to be developed, can also be used.

The invention includes a method of isolating a KDR⁺ stem cell giving rise to at least one of a mesenchymal cell, a skeletal muscle cell, a hepatic oval cell, a neuronal cell, a glial cell, a lung epithelial cell, a gastrointestinal epithelial cell, and a skin epithelial cell. The method comprises isolating a population of long-term repopulating HSCs (or other KDR⁺ stem cells) by selecting KDR⁺ cells from cells obtained from human hematopoietic tissue (or another tissue from which stem cells can be obtained) as disclosed herein. Recent data demonstrate that stem cells associated with the bone marrow have epithelial cell lineage capability, in that the cells gave rise to repopulating liver cells in transplanted rats (Petersen et al., 1999, Science 284:1168-1170). Similarly, Ferrari et al. (1998, Science 279:1528-1530), demonstrated that unfractionated bone marrow cells, when injected into recipient muscle, migrated to sites of muscle damage, and gave rise to marrow-derived cells which underwent myogenic differentiation and participated in regeneration of damaged muscle fibers. Bone marrow cells have the potential to differentiate to lineages of mesenchymal tissues, including bone, cartilage, fat, tendon, muscle, and marrow stroma (Pittenger et al., 1999, Science 284:143-147). In mice, adult bone marrow cells that were transplanted intraperitoneally migrated into the mouse's brain and differentiated to become cells that expressed neuron-specific antigens (Mezey et al., 2000, Science 290:1779-1782). Intraperitoneal transplantation of murine bone marrow cells also led to generation of both microglia and macroglia in the brain of the recipient mouse (Eglitis et al., 1997, Proc. Natl. Acad. Sci. USA 94:4080-4085). Furthermore, murine HSCs that were transplanted into primary recipients were re-transplanted into secondary recipients and gave rise to hematopoietic cells and epithelial cells of liver, lung, gastrointestinal tract, and skin tissues (Krause et al., 2001, Cell 105:369-377). Thus, mesenchymal, hepatic, myogenic, neuronal, glial, lung epithelium, gastrointestinal epithelium, and skin epithelium progenitors can be recruited from marrow-derived or other KDR⁺ cells. Without wishing to be bound by any particular theory of operation, the stem cells which gave rise to the mesenchymal, hepatic, myogenic, neuronal, glial, and epithelial progenitor cells described herein and in experiments described in the art are believed to be the cells described herein as long-term repopulating KDR⁺ stem cells. Thus, by isolating KDR⁺ stem cells as disclosed herein, it is possible to derive cells that exhibit the capability of differentiating to become one or more of a mesenchymal cell, a hepatic oval cell, a muscle cell, a neuronal cell, a glial cell, or an epithelial cell of the lung, gastrointestinal tract, or skin.

The invention includes a method of generating a differentiated human cell of a selected type from an undifferentiated HSC or from any other undifferentiated KDR⁺ stem cell. The HSC is KDR⁺ and can, for example, be isolated using the methods disclosed herein for isolating a CD34⁺KDR⁺ HSC. The KDR human stem cell is induced to differentiate into a cell of the desired type by maintaining it in the presence of a differentiated mammalian (e.g., human or murine) cell of the selected type. After a period of hours or days (typically two or three days), the KDR⁺ stem cell differentiates to become a cell of the selected type. As the examples demonstrate, when human KDR⁺ stem cells are injected into a blastocyst, they are incorporated into the developing embryo and are capable of differentiating into cells of tissues such as hematopoietic tissues, heart (myocardial) tissue, skeletal muscle tissue, gut tissue, hepatic tissue, kidney tissue, brain tissue, and spinal cord tissue. KDR⁺ HSCs injected into damaged skeletal muscle differentiate into skeletal muscle cells which are incorporated into muscle tissue, thereby repairing damaged tissue.

These data indicate that KDR⁺ HSCs, when placed in the presence of a differentiated cell other than a hematopoietic cell (i.e., in a cellular environment which supports cell differentiation), are able to differentiate to become cells of a type other than hematopoietic cells, and become incorporated into functioning, post-natal, non-hematopoietic differentiated tissue. For example, CD34⁺KDR⁺ cells that are exposed to a skeletal muscle cell are capable of differentiating into skeletal muscle cells. Of course, KDR⁺ cells can be induced to differentiate into hematopoietic and blood cells by maintaining them in the presence of a differentiated hematopoietic or blood cell. Without being bound by any particular theory, it is believed that once KDR⁺ stem cells are placed in the presence of a differentiated cell, such as a skeletal muscle cell (especially when the muscle cell is damaged, although this is not necessary), the stem cells are exposed to factors associated with cell differentiation and maintenance of a differentiated phenotype (i.e., growth factors, cell-cell signaling, and extracellular matrix attachments). The stem cells respond to these factors and differentiate, for example into skeletal muscle cells if the stem cells were placed in the presence of skeletal muscle cells. Likewise, it is believed that, when placed in the presence of another selected differentiated cell (especially when the cell is damaged, although this is not necessary), a stem cell is capable of differentiating into the other selected cell phenotype.

Purified HSCs (and other KDR⁺ stem cells) are useful in a variety of clinical settings. For example, HSCs (or other KDR⁺ stem cells) can be used as delivery vehicles for the administration of nucleic acid which is a therapeutic product or a nucleic acid encoding a therapeutic product (i.e., an RNA or protein molecule) to a human. For example, HSCs can be transfected/transduced with a suitable nucleic acid, preferably operably linked to a suitable promoter/regulatory sequence, wherein when the nucleic acid is expressed in the HSCs, a therapeutic RNA or protein is produced which is of benefit to the human. Delivery of a nucleic acid to HSCs (or other KDR⁺ stem cells) is accomplished using standard technology, for example, using viral gene transfer, described, for example, in Verma et al. (1997, Nature 389:239-242).

HSCs (or other KDR⁺ stem cells) comprising an isolated nucleic acid can be introduced into the circulating blood by intravenous injection or infusion, intraperitoneal injection or infusion, and even by intrauterine injection or infusion. Following delivery of HSCs to the circulating blood, they home to bone marrow microenvironmental niches.

Therapeutic nucleic acids which are suitable for introduction into HSCs and other KDR⁺ stem cells include a nucleic acid encoding adenosine deaminase, or a biologically active fragment thereof, for treatment of severe combined immunodeficiency, the gene encoding beta-globin, or a biologically active fragment thereof, for treatment of beta-thalassemia or sickle cell anemia, a nucleic acid comprising an antisense HIV sequence, for example, an anti-tat nucleic acid sequence, for treatment of HIV infection, a nucleic acid encoding a multi-drug resistance gene to facilitate drug resistance in transfected cells during treatment of neoplasia, and the like. Suitable promoter/regulatory sequences include the retroviral LTR and the cytomegalovirus immediate early promoter.

The invention also includes a blood substitute comprising the progeny cells derived from an isolated purified population of long-term repopulating human HSCs as described in the experimental examples that follow. The blood substitute can comprise multipotent, oligopotent, and/or unipotent progenitors. It can also comprise one or more of red blood cells, neutrophilic granulocytes, eosinophilic granulocytes, basophilic granulocytes, monocytes, and platelets, among other cells and/or components of normal blood. The blood substitute can comprise one or more of dendritic cells, T lymphocytes, B lymphocytes, and NK cells. The physiological functions of the blood substitute described herein comprise the long-term repopulating HSC which permanently and completely reconstitute the hematopoietic system of a myeloablated host, differentiated/differentiating progeny generated from the cell(s) described herein by ex vivo manipulation procedures yielding multipotent, oligopotent, and/or unipotent progenitors, or terminal differentiated cells of the erythroid, granulocytic, monocytic, dendritic/antigen-presenting cells, megakaryocytic, T- and B-lymphoid, and natural killer (NK) cell series. These blood elements function as oxygen carriers (erythroid elements), phagocytes protecting the organism against infection (neutrophilic, eosinophilic, basophilic, granulocytes and monocytes/macrophages), producers of immunoglobulins (plasma cells/B-lymphocytes; humoral immunity) which react with particular antigens, antigen-recognizing cells (T-cells; cell-mediated immunity), antigen-presenting cells (such as dendritic cells which process antigens intracellularly to peptides and present them together with MHC Class I or II molecules to CD8 and CD4 T-lymphocytes, respectively), cells killing other cells directly or by antibody-dependent cell-mediated cytotoxicity (ADCC) which they recognize as foreign (NK cells, lymphokine-activated killer cells, i.e., LAK cells), and producers of platelets (megakaryocytes) which play a central role in the haemostatic response to vascular injury.

The invention also includes a method of obtaining a purified population of long-term repopulating human HSCs that are CD34⁻lin⁻KDR⁺ (lineage marker negative), as these are defined by the examples. The method comprises obtaining a population of CD34⁻lin⁻ cells and isolating a KDR⁺ population therefrom. CD34⁻lin⁻KDR⁺ cells comprise another population comprising long-term repopulating human HSCs. CD34⁻lin⁻KDR⁺ cells can convert to their CD34⁺ counterparts in vivo as CD34⁻lin⁻ cells convert into CD34⁺lin⁻ cells in vitro (Zanjani et al., 1998, Blood (Suppl. I) 92:504). Therefore, a purified population of long-term repopulating HSCs can be obtained by first selecting for CD34⁻lin⁻, by FACS or by use of immunobeads as described herein for isolation of CD34⁺ cells, and then further selecting from the CD34⁻lin⁻ population the sub-fraction of KDR as described herein. The use of antibodies specific for human cell markers to obtain purified populations of cells is known in the art and is described herein. Other methods known in the art for separation of cell subsets, or methods to be developed, can also be used to practice the present invention, as discussed for CD34⁺ and CD34⁺KDR⁺ cell populations. Long-term repopulating CD34⁻lin⁻KDR⁺ HSCs can be used for similar purposes, and in similar ways to the CD34⁺KDR⁺ HSCs described herein, for example, as a blood substitute, for administration of a nucleic acid which is therapeutic, and/or in transplantation medicine.

The invention includes a chimeric mammal engrafted with at least one of an isolated purified long-term repopulating human HSC (or other KDR⁺ stem cell). That is, the invention includes a mammal that has received an HSC or other KDR⁺ stem cell from another mammal or an autologous transplant wherein the stem cell is reintroduced into the mammal after being isolated and purified from that same mammal by ex vivo methods, such as those described herein. Thus, stem cells isolated from a mammal can be re-introduced into the same mammal, or into a different mammal, optionally after an exogenous nucleic acid has been introduced into the cell. The present invention encompasses the introduction of a nucleic acid into a mammal by the process of introducing an isolated nucleic acid into a KDR⁺ stem cell removed from that animal and using the stem cell to engraft the animal. The stem cells can be isolated from the same recipient animal or it can be obtained from another donor animal of the same, or a different, species. However, the invention is not limited to this method of producing a chimeric animal. Instead, the invention encompasses the production of a chimeric animal by other methods known in the art, such as blastocyst injection.

The introduction of an isolated nucleic acid into an HSC (or other KDR⁺ stem cell) has been described herein, and the methods for expanding the HSCs, for introducing them, and thereby engrafling an animal with the cells are described herein. One skilled in the art, based upon the disclosure provided herein, is able to generate a chimeric mammal engrafted by at least one isolated repopulating HSC by intravenous transfusion into the animal. However, any other method of delivering repopulating HSCs to mammal recipients can be used. Further, the recipient animal's hematolymphopoietic system can be either ablated before engraftment of the cell(s), or the cell(s) are introduced into the animal in addition to the animal's own hematopoietic system.

Hematopoietic multilineage engraftment in the recipient mammal is defined as permanent and complete (i.e., reconstitution of all hematopoietic lineages through donor HSCs), as well as sustained production of HPCs. Multilineage engraftment is detectable using specific MoAbs recognizing cells pertaining to a particular lineage. As an example, erythroid cells are recognized by anti-glycophorin A (GPA) MoAb, megakaryocytes (MKs) are recognized by MoAbs such as anti-CD61 and anti-CD41, and HPCs are recognized by clonogenic assay and anti-CD34 MoAbs, anti-AC133 MoAbs, and the like.

The invention includes a method of inhibiting rejection of a transplanted organ. The method comprises engrafling the organ recipient using an isolated and purified long-term repopulating human HSC (or other KDR⁺ stem cell) obtained from the organ donor prior to transplanting the organ. The bone marrow of the recipient is ablated by standard methods known in the art. Generally, bone marrow ablation is accomplished by X-radiating the animal to be transplanted, administering drugs such as cyclophosphamide, or by a combination of X-radiation and drug administration. In some embodiments, bone marrow ablation is produced by administration of radioisotopes known to kill metastatic bone cells, for example, radioactive strontium, ¹³⁵Samarium, or ¹⁶⁶Holmium (Applebaum et al., 1992, Blood 80:1608-1613). By engrafting the hematopoietic system of the recipient with HSCs from the organ donor, rejection of the transplanted organ is inhibited.

Similarly, the invention includes a method of transplanting an autologous human HSC (or other KDR⁺ stem cell) in a human. The method comprises isolating a population of long-term repopulating stem cells from the recipient and ablating the bone marrow of the recipient. Non-malignant long-term repopulating stem cells are isolated by selecting KDR⁺ cells as disclosed herein. Non-malignant cells are identified within a population of KDR⁺ cells based on various criteria known in the art including cell morphology, biochemical properties, growth characteristics, and expression of specific tumor cell markers. Thus, the bone marrow of the individual is purged of malignant blasts and other malignant cells such that by transplanting the non-malignant stem cells back into to the individual, diseases such as melanomas can be treated. That is, for diseases where the malignant cells do not express KDR, the bone marrow can be ablated and cells previously obtained from the individual can be enriched for non-malignant long-term repopulating HSCs and returned to the patient where they cause multi-lineage engraftment, thereby treating or alleviating the disease. Any disease, disorder, or injury associated with irreversible tissue damage can be inhibited, prevented, alleviated, reversed, or cured by administering HSCs to the tissue affected by the disease, disorder, or injury.

HSCs (or other KDR⁺ stem cells) can be administered to a tissue and allowed to differentiate in vivo. Administration of stem cells to a tissue can be accomplished by local injection of stem cells to the tissue, systemic administration via the bloodstream, or other methods known in the art. Preferably, at least about 100-1,000 KDR⁺ stem cells, initially differentiated stem cells (i.e., stem cells committed to a particular cell lineage, such as an epithelial or endothelial cell lineage), or tissue-specific progenitor cells are administered, although smaller numbers can suffice, particularly if they are locally administered to the desired tissue. The stem cells can be expanded ex vivo prior to administration to the tissue. Alternatively, or in addition, the stem cells can be differentiated to give rise to tissue-specific stem cells or progenitor cells prior to administration to the tissue. Alternatively, the stem cells can be expanded, differentiated, or both, in a donor subject, isolated from the donor's tissue, and then administered to a recipient's tissue. For example, human KDR⁺ stem cells can be injected into a non-human mammalian blastocyst, embryo, or fetus, wherein the human stem cells are incorporated into tissues of the developing blastocyst, embryo, or fetus, and can at least begin to differentiate therein (e.g., to yield tissue-specific stem cells or progenitor cells). When the cells are injected into a blastocyst, as few as 30 or 40 cells can be used. When the cells are injected into an embryo or fetus, injection of a larger number of cells (preferably hundreds, thousands, or tens of thousands) is desirable. Expanded and/or differentiated human cells (e.g., KDR⁺ stem cells such as HSCs or human mesenchymal, neuronal, myogenic, glial, or epithelial cell progenitors) can be isolated from tissues of the non-human mammal shortly after incorporation or at a more advanced developmental stage of the mammal (e.g., post-natally) and administered to a tissue of the human. Stem cells can be isolated from regions in which they are known to concentrate (e.g., the embryonic aorta-gonad-mesonephros region; Labastie et al., 1998, Blood 92(10):3624-3635), from an embryonic or fetal hematopoietic tissue, or from homogenized embryonic or fetal cell preparation, for example.

Initially differentiated stem cells or particular tissue progenitor cells can be isolated from tissues in which their occurrence is known or expected. The method used to separate the human and non-human cells is not critical. By way of example, human cells can be separated from the non-human cells of the mammal using standard cell-sorting methods and reagents (e.g., antibodies) that are specific for the desired human cell type. In this manner, the non-human mammal can provide a one-time or continual supply of differentiated human cells. Without being bound by any particular theory of operation, it is believed that differentiation of human stem cells and tissue progenitor cells is influenced by cytokines released from specific tissues and by the cell-to-cell and cell-to-matrix interactions in the mammal.

For example, stroke leads to localized death of CNS cells in a brain region to which normal blood supply is inhibited or interrupted, resulting in irreversible tissue damage. Following a stroke, CNS cells normally do not repopulate the affected area, and neural, sensory, cognitive, and motor defects can result from the loss of brain cells. Injection of HSCs (or other KDR⁺ stem cells) to the affected CNS area places the stem cells in an environment in which CNS cell differentiation is induced, supported, or both. Stem cells placed in this CNS environment differentiate into CNS cells which repopulate the affected area, restoring neuronal and neural (generally, e.g., neuron-glial) interconnections and normal CNS functions.

Similarly, myocardial injury caused by diseases or disorders such as chronic coronary disease or myocardial infarction, for example, results in irreversibly damaged myocardial tissue and diminished cardiac function. Injection of HSCs (or other KDR⁺ stem cells) into an affected myocardial area places the HSCs in an environment in which myocardial and endothelial cell differentiation is induced, supported, or both, resulting in differentiation of HSCs into myocardial and endothelial cells and restoration of normal cardiac function.

Other diseases, disorders, or tissue injuries that can be treated by injection and differentiation of HSCs (or other KDR⁺ stem cells) include

i) spinal cord injury (to replace damaged nerve cells, glial cells, or other damaged CNS cells, for example);

ii) multiple sclerosis (to replace damaged nerve cells, for example, or to replace a subject's immune system, thereby eliminating auto-reactive immune cells, for example);

iii) Alzheimer's disease (to replace nerve cells, for example);

iv) Parkinson's disease (to replace damaged nerve cells, for example);

v) liver fibrosis (to replace damaged liver cells, for example);

vi) liver cirrhosis (to replace damaged liver cells, for example);

vii) chronic obstructive pulmonary disorder (to replace damaged lung cells, for example);

viii) compartment syndrome (to replace bone cells, muscle cells, nerve cells, or endothelial cells, for example);

ix) chronic inflammation or chronic infection (to replace necrotic tissue at the site of the chronic inflammation or chronic infection, for example); and

x) wound healing (to replace damaged epithelial or connective tissue, for example, or to reduce scar formation after surgery, for example).

The methods described herein for generating a differentiated cell from an undifferentiated HSC (or other KDR⁺ stem cell) can be used to rejuvenate tissue that has suffered age-related damage which results in, for example, a decline in tissue function or altered appearance of the tissue. During the aging process, cells experience diminished biochemical functions, decreased cellular replication capacity, and accumulate cytotoxic insults (such as oxidative stress, ultraviolet light, or chemical exposure). An example of a tissue that is susceptible to age-related damage is skin, which incurs damage that results in loss of skin elasticity and skin thinning. Skin also experiences an age-related decline in cell replacement capacity. Therefore, in an older subject, skin is a tissue that can be rejuvenated (i.e., made to look, feel, and function more like the same tissue of a younger subject), by providing HSCs (or other KDR⁺ stem cells) to the skin of the older subject. Stem cells provided to the skin are capable of differentiating into cells that comprise skin tissue (such as keratinocytes, fibroblasts, melanocytes, Langerhan's cells, and the like), thereby enhancing the appearance of the skin. Other cells and tissues that suffer age-related damage that can be rejuvenated, repaired, or regenerated using the methods described herein include liver tissue, kidney tissue, retinal tissue, lens epithelia, lens fibers, muscle tissue, connective tissue, immune cells, brain tissue, and nerve tissue.

Tissues that have been damaged by wounding (e.g., as a result of physical trauma, surgical incision, or surgical resection) can be repaired, restored, or re-grown according to the methods of the invention. Likewise, muscle tissue that has atrophied as a result of a body part having been in a cast, extensive bed rest, or paralysis, for example, can be enhanced, rebuilt, and strengthened by providing HSCs (or other KDR⁺ stem cells) to the affected muscle tissue.

HSCs and other KDR⁺ stem cells that are isolated from a tissue can be stored for later use, as can stem cells that have been induced to differentiate into tissue-specific stem cells or progenitor cells. A variety of methods are known for storing stem cells, and the method disclosed by Rubinstein et al., (1995, Proc. Natl. Acad. Sci. USA 92:10119-10122) is one desirable method of storing these cells. Alternatively, KDR⁺ stem cells can be isolated and expanded ex vivo as described herein, and stored for later use. For example, a parent can elect to have HSCs isolated from a newborn's cord blood and stored for later use. Alternatively, the cord blood-derived HSCs can be expanded ex vivo prior to storage. The stored HSCs from a newborn's cord blood can be used to inhibit, prevent, alleviate, reverse, or cure a disease or disorder associated with irreversibly damaged tissue. Furthermore, because of the plasticity that KDR⁺ stem cells exhibit, a person can elect to have their own HSCs isolated, as described herein, from a hematopoietic tissue (e.g., bone marrow) for storage, or for ex vivo expansion followed by storage. The stored HSCs provide a readily available source of undifferentiated stem cells with a significant potential for differentiation into any desired cell phenotype. Throughout the person's life, tissue damage can be inhibited, prevented, alleviated, reversed, or cured using the stored KDR⁺ stem cells. Additionally, the stored KDR⁺ stem cells can be used to rejuvenate the person's aged tissues.

The invention includes a method of monitoring the presence of KDR⁺ stem cells in a human hematopoietic tissue in a human receiving therapy. The method comprises obtaining a hematopoietic tissue sample from the human and measuring the number of KDR⁺ stem cells in the sample. Measurements are made before, during and after therapy where therapy can be chemotherapy and/or radiation therapy which is known to affect the stem cell compartment, for example, myeloablation therapy or therapy known to cause hematopoietic suppression. Until the present invention, no method was available to allow the status of the stem cell compartment to be determined during such therapy. The present invention, by defining a marker, i.e., KDR⁺, for the cells of this compartment, allows the determination of the status of the stem cell compartment in a patient receiving therapy known or thought to affect the stem cell compartment at any point before, during, and after therapy.

Definitions

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“Antibody,” means an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention can exist in a variety of forms including, for example, polyclonal antibodies and humanized antibodies (Harlow et al., 1999, In: Using Antibodies: A Laboratory Manual, Cold Spring Harbor, New York; Harlow et al., 1988, In: Antibodies: A Laboratory Manual, Cold Spring Harbor, New York; Houston et al.,1988, Proc. Natl. Acad. Sci. USA 85:5879-5883; Bird et al., 1988, Science 242:423-426).

“Specifically binds” means binding between a molecule and a ligand of that molecule (e.g., an antibody which recognizes and binds CD34 polypeptide), wherein the molecule does not substantially recognize or bind other molecules in a sample. For example, an antibody “specifically binds KDR” if the antibody recognizes and binds VEGFR2/KDR/flk-1 in a sample but does not substantially recognize or bind to other molecules in a sample. Further, an antibody specifically binds lin markers if the antibody recognizes and binds lineage markers but does not substantially recognize or bind to other molecules in a sample.

“Synthetic antibody,” means an antibody which is generated using recombinant DNA technology, for example, an antibody expressed by a bacteriophage as described herein. The term includes an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and known in the art.

“Antisense nucleic acid” means a nucleic acid polymer, at least a portion of which is complementary to another nucleic acid. The antisense nucleic acid can comprise between about fourteen and about fifty or more nucleotides. Preferably, the antisense nucleic acid comprises between about twelve and about thirty nucleotides. More preferably, the antisense nucleic acid comprises between about sixteen and about twenty-one nucleotides. The antisense nucleic acid can include phosphorothioate oligonucleotides and other modifications of oligonucleotides. Methods for synthesizing oligonucleotides, phosphorothioate oligonucleotides, and otherwise modified oligonucleotides are known in the art (U.S. Pat. No: 5,034,506; Nielson et al., 1991, Science 254:1497).

“Antisense” refers particularly to the nucleic acid sequence of the non-coding strand of a double stranded DNA molecule or, in the case of some viruses, a single or double stranded RNA molecule, encoding a protein, or to a sequence which is substantially homologous to the non-coding strand. It is not necessary that the antisense sequence be complementary solely to the coding portion of the coding strand of the nucleic acid molecule. The antisense sequence can be complementary to regulatory sequences specified on the coding strand of a nucleic acid molecule encoding a protein, which regulatory sequences control expression of the coding sequences.

“Sense” refers to the nucleic acid sequence of the single or double-stranded nucleic acid molecule which encodes a protein, or a sequence which is substantially homologous to that strand. However, the nucleic acid sequence is not limited solely to the portion of the coding strand encoding a protein; rather, the sequence can include regulatory sequences involves in, for example, control of expression of the coding sequence.

“Biochemical/biological property” means any biochemical/biological property of a cell which allows the purification of such cell. A biochemical/biological property includes, for example, the ability of a cell to take up or to exclude certain dyes.

“Blood substitute” means a substance derived from long-term repopulating human HSCs comprising at least one component of naturally-occurring blood for example, red blood cells, platelets, and other components/products of normal blood. Further, the blood substitute refers to a substance that can perform at least one of the biochemical/physiological functions of normal blood such as the transport of oxygen, and the like.

“chimeric mammal” is any mammal which is a recipient of at least one long-term repopulating human HSC from another mammal.

“Complementary” refers to the broad concept of subunit sequence complementary between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs).

“Coding” and “encoding” mean that the nucleotide sequence of a nucleic acid is capable of specifying a particular polypeptide of interest. That is, the nucleic acid can be transcribed and/or translated to produce the polypeptide. Thus, for example, a nucleic acid encoding adenosine deaminase is capable of being transcribed and/or translated to produce an adenosine deaminase polypeptide.

“Co-expressed” means that the antigen is expressed on or in a cell which also comprises detectable KDR antigen. However, the two molecules need not be co-expressed contemporaneously. Rather, it is sufficient that the cell express both KDR and the co-expressed antigen at some point in time such that selection of a cell expressing the other antigen selects for cells which either at that moment, or at some later time, also express KDR.

An “early marker” is any antigen on the surface of a cell which is preferentially or selectively expressed on the surface of undifferentiated precursor cells compared to its expression on the surface of differentiated cells. Examples of early markers for hematopoietic cells include CD34, Thy-1, c-kit receptor, flt3 receptor, AC133, VEGF receptor I, VEGF receptor III, Tie1, Tek, and basic fibroblast growth factor receptor.

“Engrafted” means that the mammal comprises a hematolymphopoietic system repopulated by multi-lineage cells derived from at least one isolated purified HSC which was administered to the animal.

“Enriched” means that a population of cells comprises a detectably higher level of the enriched cell type than an otherwise identical cell population not subjected to selection for that cell type. The level of enrichment can be determined by comparing the number of cells of interest in an unselected population to the number of cells of interest in a population selected for a particular trait or marker by a cell selection method.

“Isolated” refers to a cell that has been selected from other cells based on a specific characteristic (e.g., expression of a cell surface marker, cell shape, cell size, and the like). Likewise, “isolating” refers to the process of selecting a cell from other cells based on a specific characteristic. For example, an “isolated KDR⁺ cell” is a cell that has been selected out of a population or group of other cells on the basis of KDR expression. A population of “isolated cells” is comprised of a group of similar cells that have been selected from a larger, heterogeneous group of cells based on a specific characteristic. For example, a population of isolated stem cells is a group of cells that has been selected from a larger group of cells comprising stem cells and non-stem cells, based on stem cell characteristics, such as those described herein. Isolated KDR⁺ cells are preferably present in a population that is at least about 80% KDR⁺ cells, and more preferably at least 90% KDR⁺ cells. Typically, a population of cells referred to herein as isolated KDR⁺ cells means a population in which about 80% to 100% of the cells express the KDR marker. Although cell populations comprising fewer than 80% KDR⁺ cells can be used in the methods described herein, the efficiency of the methods will generally decrease as the proportion of KDR⁺ cells in the population decreases.

An “isolated nucleic acid” is a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

“KDR⁺” means that a cell expresses detectable KDR antigen. The antigen can be detected by a variety of methods including PCR, RT-PCR, Western blotting, and immunofluorescence. With regard to immunofluorescence, KDR⁺ cells can be designated KDR⁺ (i.e., KDR^{bright}) and KDR^{+/±} (i.e., KDR^{dim}) when stained using an anti-KDR monoclonal antibody, such as MoAb 260.4 under the conditions disclosed herein.

A “late marker” is a marker associated with or preferentially expressed on differentiated precursor cells. Such markers are known in the art and include the lineage (lin) markers (e.g., CD2, CD4, CD8, CD13, CD14, CD15, CD16, CD19, Cd20, CD33, CD45RA CD61, GPA, Gr-1, B220, and the like; Gabbianelli et al., 1990, Science 249:15611-1564; Sposi et al., 1992, Proc. Natl. Acad. Sci. USA 89:6353-6357; Giampaolo et al., 1994, Blood 84:3637-3647; Labbaye et al., 1995, J. Clin. Invest. 95:2346-2358; Goodell et al., 1996, J. Exp. Med., 183:1797-1806; Bhatia et al., 1998, Nature Med. 4:1038-1045; Ziegler et al., 1999, Blood 93:3355-3368).

A “multi-lineage engrafting dose” is at least one long-term repopulating human HSC which, when transplanted into an animal, is capable of giving rise to detectable multi-lineage engraftment of the recipient animal.

“Non-malignant” means that a cell does not exhibit any detectable traits typically associated with neoplastic cells such as the loss of contact-inhibition, and the like.

A “physical property” is any property of a cell which can be used to physically isolate such cell. For example, physical properties of a cell include cell size, cell density, cell mass, and cell morphology.

A “promoter/regulatory sequence” is a DNA sequence which is required for expression of a gene operably linked to the promoter/regulator sequence. In some instance, this sequence can be the core promoter sequence and in other instances, this sequence can also include an enhancer sequence and other regulatory elements which are required for expression of the gene in a tissue-specific manner.

By describing two nucleic acid sequences as “operably linked,” as used herein, is meant that a single-stranded or double-stranded nucleic acid moiety comprises each of the two nucleic acid sequences and that the two sequences are arranged within the nucleic acid moiety in such a manner that at least one of the two nucleic acid sequences is able to exert a physiological effect by which it is characterized upon the other.

“Starvation-resistant” means that a cell has the ability to survive at least about 5-10 days (shorter starvation times may apply) in liquid suspension culture in FCS-free and serum-free medium (or any other type of suitable medium) in absence of added HGFs, except VEGF, under the conditions described herein.

“Transfected” or “transduced” mean any method by which an isolated nucleic acid can be introduced into a cell. Such methods are known in the art and are described in, for example, Sambrook et al. (1989, Molecular Cloning: A Laboratory Manual, Cold Spring Harbor Laboratory Press, New York) and Ausubel et al. (1997, Current Protocols in Molecular Biology, Green & Wiley, New York). For instance, the nucleic acid can be introduced into a cell using a plasmid or viral vector, electroporation, a “gene gun,” polylysine compounds, and the like.

“Vector” means any plasmid or virus encoding an exogenous nucleic acid. The term includes non-plasmid and non-viral compounds which facilitate transfer of nucleic acid into virions or cells, for example, polylysine compounds and the like. The vector can be a viral vector which is suitable as a delivery vehicle for delivery of the isolated nucleic acid of interest (e.g., adenosine deaminase, beta-globin, multi-drug resistance, and the like) to a cell, or the vector can be a non-viral vectors which is suitable for the same purpose. Examples of viral and non-viral vectors for delivery of nucleic acids to cells and tissues are known in the art and are described, for example, in Ma et al. (1997, Proc. Natl. Acad. Sci. USA 94:12744-12746). Examples of viral vectors include a recombinant vaccinia virus, a recombinant adenovirus, a recombinant retrovirus, a recombinant adeno-associated virus, a recombinant avian pox virus, and the like (Cranage et al., 1986, EMBO J. 5:3057-3063; International Patent Application No. WO94/17810, published Aug. 18, 1994; International Patent Application No. WO94/23744, published Oct. 27, 1994). Examples of non-viral vectors include liposomes, polyamine derivatives of DNA, and the like.

The invention will be further described by reference to the following experimental examples. These examples are provided for purposes of illustration only, and are not limiting unless otherwise specified. Thus, the invention is not limited to the following examples, but rather, encompasses any and all variations which are evident as a result of the teaching provided herein.

### EXAMPLES

The experiments which are presented herein examine the expression and functional role of VEGFR, particularly the VEGFRII termed flk1/KDR, in HPCs/HSCs purified from embryonic-fetal liver (FL), cord blood (CB), normal or mobilized adult peripheral blood (PB, MPB) and adult bone marrow (BM). As indicated herein, these purified lin⁻(lineage marker negative) HPC populations comprise a small minority of HSCs. The data are summarized as follows.

### Example 1

KDR expression on purified CD34⁺ HPC populations was analyzed using a monoclonal antibody (MoAb) which recognizes the extracellular receptor domain.

MoAb evaluation indicated that KDR is expressed on approximately <1% CB, BM, PB or MPB CD34⁺ cells under the conditions used herein. Representative results using this MoAb indicated that KDR is expressed on approximately ≧1% FL CD34⁺ cells. Without wishing to be bound by theory, other antibodies and/or varying detection conditions can affect the percentage of KDR⁺ cells detected in a CD34⁺ population of cells.

KDR expression is virtually restricted to adult and CB HSCs and a portion of the most primitive subset of adult and CB HPCs. KDR is also expressed on approximately <1% of CD34⁻lin⁻ cells.

The KDR⁺ versus KDR⁻ cell fractions were sorted from CD34⁺ HPCs purified from CB, BM, PB or MPB. In both cell fractions, the following assays were performed: (i) Assay of HPCs in clonogenic culture; (ii) assay of long-term repopulating HSCs in vitro (i.e., evaluation in 12 week LTC of the frequency of CAFCs and/or LTC-ICs: the frequency was evaluated by limiting dilution assay (LDA)) and in vivo, i.e., analysis of hematopoietic repopulation in NOD-SCID mice at 3 months after sub-lethal irradiation and cell injection. The results consistently established that the CD34⁺KDR⁺ and/or the CD34⁺ KDR^{+/±} fraction contained little or no uni-oligopotent HPCs, and a minority of multipotent and primitive HPCs, whereas it was dramatically enriched for HSCs. Conversely, the CD34⁺KDR⁻ fraction contained virtually all uni-oligopotent HPCs, as well as multilineage and primitive HPCs, and essentially no long-term repopulating HSCs.

In clonogenic semisolid culture, treatment of CD34⁺KDR⁺ cells with VEGF, combined with diverse cocktails of hematopoietic growth factors (HGFs), caused a mild stimulatory effect on multipotent HPCs and primitive HPCs. More importantly, LDA of LTC-IC/CAFC frequency in the KDR⁺ and KDR⁻ cell fraction from PB, BM, or CB in Dexter type 12 week LTC revealed that, in PB, BM and CB KDR⁺ cell fractions, the LTC-IC/CAFC frequency was elevated (approximately ≧50-60%, representative results) in LTC supplemented with VEGF, whereas it was lower (approximately 25-43%, representative results) in PB, BM and CB LTC which were not supplemented with VEGF. In both BM and CB KDR⁻ cell fractions, the LTC-IC/CAFC frequency was 0% or close to 0% with or without VEGF treatment. Similar results on LTC-ICs/CAFCs were obtained in MPB KDR⁺ cells. In preliminary experiments, twelve week incubation of normal PB KDR⁺ cells with VEGF in single cell LTC, followed by seeding the generated cells into secondary LTC, caused an amplification of the number of HSCs, assayed as 12 week LTC-ICs. In addition, liquid suspension culture experiments on CD34⁺KDR⁺ versus CD34⁻KDR⁻ CB cells confirmed that only the KDR⁺ cell fraction generated in the long-term (approximately 12 week culture) primitive CD34⁺CD38⁻ HPCs, particularly when stimulated by not only early acting HGFs (see below) but by VEGF combined with early acting HGFs. CD34⁺KDR⁺ cells seeded in single cell or minibulk FCS⁻ free HGF⁻ starvation culture partially survived for up to at least 1 month upon addition of VEGF. The starvation-resistant cells were enriched for putative HSCs (up to approximately ≦80-95%, representative results).

These data therefore establish the following. VEGFRII (KDR) expression is restricted to a small subset of CB, BM, PB and MPB CD34⁺ HPCs. This subset comprises virtually no uni- or oligopotent HPCs, a fraction of primitive HPCs and virtually the entire pool of long-term repopulating CD34⁺ HSCs, respectively endowed with modest or extensive self-renewal capacity. Consistent with these results, VEGF selectively stimulates the proliferation of and/or protects against apoptosis primitive HPCs and particularly HSCs.

Furthermore, preliminary experiments suggest that KDR⁺ cells in the CD34⁻lin⁻ cell population purified from adult hematopoietic tissues also contained a fraction of long-term repopulating HSCs. Therefore, the data disclosed herein demonstrate that KDR is novel key marker for human long-term repopulating HSCs and that the VEGF/KDR system plays a key role in long-term HSC function.

The Materials and Methods used in the experiments presented herein are now described.

VEGFRII (KDR) antibody

The mouse monoclonal antibody (clone 260.4), raised against the KDR soluble protein and recognizing the extracellular KDR domain, was obtained from Gesellschaft für Biologische Forschung, GBF, Braunschweig, Germany.

Hematopoietic growth factors (HGFs)

Recombinant human HGFs were purchased from commercial sources (see below); VEGF was purchased from R&D Systems (Minneapolis, Minn.).

Cells and purification procedures

Human HPCs (containing a small HSC sub-population), and the KDR⁺ fraction thereof, were purified from (i) fetal liver (FL), (ii) cord blood (CB), (iii) adult bone marrow (BM), and (iv) adult normal or mobilized peripheral blood (PB, MPB), as described below.

CD34⁺ cell purification

BM cells were obtained from consenting normal donors. MPB was obtained from G-CSF-treated (5 micrograms per kilogram per day) consenting normal donors. Normal PB was collected as buffy coat preparation from the local blood bank. CB was obtained from healthy, full-terrn placentas according to institutional guidelines. Low-density cells (<1.077 grams per milliliter) were isolated by Ficoll and CD34⁺ cells purified by MiniMACS column (Miltenyi Bergisch Gladbach, Germany and Auburn, Calif.).

Fluorescence staining and flow cytometry analysis

Purified CD34⁺ cells were incubated for 30 minutes on ice with saturating amounts of biotinylated anti-KDR MoAb (clone 260.4, Gesellschaft für Biologische Forschung, Braunschweig, Germany) and anti-CD34 FITC MoAbs (clone HPCA-2, Becton-Dickinson (B-D), San Jose, Calif.). For three color FACS analysis, anti-CD34 PerCP and one of following FITC-conjugated MoAbs were used: anti-CD38 (B-D), anti-flt3 (Immunotech, Marseille, France), anti-Thy-1 (Pharmingen, San Diego, Calif.), anti-c-kit (Serotec, Oxford, UK). The cells were then washed and labeled with streptavidin-PE (B-D). After a further washing, cells were run on a FACScan™ or FACSCalibur™ FACS device for two- or three-color analysis.

CD34⁺KDR⁺ cell separation

Purified CD34⁺ cells were incubated with saturating amounts of anti-CD34-FITC and biotinylated anti-KDR, washed and labeled with streptavidin-PE (B-D). After a further washing, CD34⁺KDR⁺ or KDR^{+/±} and CD34⁺KDR⁻ sub-populations were sorted on FACSVantage™ (B-D) or EPICS™ Elite (Coulter) (fluorescence emission, 525 and 575 nanometers). A fraction of sorted KDR⁻ cells was reanalyzed: if contaminating KDR⁺ cells were detected, the population was re-stained and resorted to ensure elimination of all KDR⁺ cells.

KDR RT-PCR was performed as described using primers having the sequences 5′-AAAACCTTTT GTTGCTTTTG GA-3′(SEQ ID NO: 1) and 5′-GAAATGGGAT TGGTAAGGAT GA-3′(SEQ ID NO: 2; Ziegler et al., 1999, Science 285:1553-1558; Terman et al., 1991, Oncogene 6:1677-1683).

In Vitro Assays

HPC assay

HPCs were seeded in 0.9% methylcellulose fetal calf serum free (FCS⁻) medium supplemented with saturating amounts of HGFs (flt3 ligand {FL}, kit ligand, {KL}, basic fibroblast GF {bFGF}, 100 nanograms per milliliter each; interleukin 6 {IL6}, 10 nanograms; IL3, 100 units; granulomonocyte colony-stimulating factor {GM-CSF}, 10 nanograms; G-CSF, 500 units; M-CSF, 250 units; thrombopoietin {Tpo}, 100 nanograms, erythropoietin {Epo}, 3 units). CFU-Mix/BFU-E and CFU-GM colonies comprised >5×103 and >10³ cells, respectively (Gabbianelli et al., 1995, Blood 86:1661-1670). A more limited HGF combination comprised IL3, GM-CSF, and Epo at the indicated dosages (this culture condition was also utilized for NOD-SCID mice BM mononuclear cell (MC) clonogenic assay; Gabbianelli et al., 1995, Blood 86:1661-1670). CFU-Mix/BFU-E and CFU-GM colonies comprised >500 and >100 cells, respectively. For detection of human colonies, the colony DNA was processed for PCR using KlenTaq-1 DNA polymerase (Clontech, Palo Alto, Calif.) and primers recognizing human alpha-satellite sequences on chromosome 17 (Warburton et al., 1991, Genomics 11:324-333).

HPP-CFC assay

HPP-CFC assay was performed as described in Gabbianelli et al., (1995, Blood 86:1661-1670). Primary HPP-CFC clones, scored at day 30, were re-plated for secondary HPP-CFC colony formation.

5-, 8-, or 12-week LTC

The LTC were established on allogeneic irradiated (20 Gray) BM stromas or FBMD-1 cells (Gabbianelli et al., 1995, Blood 86:1661-1670; van der Loo et al., 1995, Blood 85:2598-2606). At weekly intervals half of the medium was removed and replaced by fresh medium ± VEGF (100 nanograms per milliliter). In 12-week LTC irradiated BM stromas or fresh FBMD-1 cells were added monthly to prevent functional exhaustion of the initial inoculum (Hao et al., 1996, Blood 88:3306-3313). In minibulk LTC, each well was seeded with 100-1,000 CD34⁺KDR⁺ cells (1,000 cells per milliliter; positive or negative control was seeded with 10,000 CD34⁺ or CD34⁺KDR⁻ cells, respectively). LTC were terminated at 5-, 8-,or 12-weeks and cells from supernatant and adherent fractions were cultured in semisolid medium for colony growth (Gabbianelli et al., 1995, Blood 86:1661-1670). Alternatively, 6-,9-, or 12-week CAFCs were scored directly in LTC adherent layer (van der Loo et al., 1995, Blood 85: 2598-2606).

Limiting dilution assay (LDA)

Graded numbers of CD34⁺KDR⁺ cells (1-100 cells per well) were seeded in LTC wells (Sutherland et al., 1990, Proc. Natl. Acad. Sci. USA 87:3584-3588; Care et al., 1999, Oncogene 18:1993-2001). The frequency of 12-week LTC-ICs/CAFCs was calculated according to single hit Poisson statistics (Sutherland et al., 1990, Proc. Natl. Acad. Sci. USA 87:3584-3588; Care et al., 1999, Oncogene 18:1993-2001). Control LDA was performed on CD34⁺KDR⁻ cells (10-5,000 cells per well) and unseparated CD34⁺0 cells (20-5,000 cells per well).

Liquid phase suspension culture

Liquid phase suspension culture in FCS⁻ medium ± VEGF and ± other HGFs was performed as in described in Ziegler et al. (1999, Blood 93:3355-3368). In the representative minibulk (2-3×10³ CD34⁺KDR^{+/±} or CD34⁺KDR⁻ cells per well) or in single cell (one CD34⁺KDR^{+/±} or CD34⁺KDR⁻ cell per well) starvation culture experiments, cells were treated only with VEGF (100 nanograms per milliliter). In a VEGF±HGFs representative experiment 1,000 purified CD34⁺KDR⁺ or CD34⁺KDR⁻CB cells were grown in 100 microliters of FCS-free medium in individual wells of a 96-well plate until cell numbers reached approximately 10,000 cells per well on or about day 14 (Gabbianelli et al., 1995, Blood 86:1661-1670). Thereafter, the cells were transferred to individual wells of a 24-well plate with 500 microliters of medium. Cultures were supplemented with VEGF (50 nanograms per milliliter) either alone or combined with Tpo (100 nanograms per milliliter), FL (100 nanograms per milliliter), IL-3 (0.1 nanogram per milliliter). HGF combinations were VEGF alone, VEGF+FL, VEGF+FL+Tpo, VEGF+FL+IL-3, and FL+Tpo+IL-3. At weekly intervals, one half of the medium was replaced by fresh medium and HGFs. Starting at day 25 of culture, cell numbers were determined weekly and immuno-phenotype analysis of cultured cells was performed weekly using anti-CD34 and anti-CD38 MoAbs. The cultures were maintained for 12 weeks.

NOD-SCID mice xenografts

Six 8-week old mice (Jackson Laboratory, Bar Harbor, Me.) were irradiated at 3.5 Gray using a ¹³⁷Cs source (Gammacell) 12-24 hours prior to xenotransplantation. KDR⁺ or KDR⁻ cells were injected by tail vein injection together with 100,000 irradiated (20 Gray) BM or CB mononuclear cells (MCs). Mice were killed 12 weeks after xenotransplantation according to institutional regulations. Cell suspensions from femurs, spleen and PB were analyzed for human cells by flow cytometry: erythrocytes depleted cells were labeled with FITC- or PE-conjugated MoAbs which specifically bound the following markers: CD45 (HLe1), CD34 (HPCA-2), CD38, CD15, CD33, CD71, CD2, CD3, CD4, CD7, CD8, CD19 CD20, CD16, CD56 (B-D); GPA, CD71 (Pharmingen, San Diego, Calif.). FITC- or PE-conjugated isotype-matched irrelevant MoAbs were used as controls. Bone marrow, spleen and PB cells from non-transplanted mice were used as negative control. Positive controls consisted of human BM or CB MCs. BMMCs were also cultured in semisolid media selective for human HPCs as described herein.

Fetal sheep xenografts

Fetal sheep xenographs were performed as described (Zanjani et al., 1998, Exp. Hematol. 26:353-360; Civin et al., 1996, Blood 88:4102-4109; Kawashima et al., 1996, Blood 87:4136-4142; Sutherland et al., 1996, Exp. Hematol. 24:795-806; Uchida et al., 1996, Blood 88:1297-1305). PB and BM MCs from chimeric fetuses/newborns, separated by Ficoll gradient, were evaluated for presence of human cells by flow cytometry. BMMCs were also assayed for human HPCs in clonogenic culture by karyotyping of hematopoietic colonies. Human CD34⁺ cells, isolated by MiniMACS column from BMMCs of primary recipients as described herein, were transplanted in secondary recipients.

Receptor-type tyrosine kinases (RTKs) RT-PCR assay in CD34⁺KDR⁺ cells

BM CD34⁺KDR⁺ cells were isolated by double sorting and analyzed by RT-PCR (Ziegler et al., 1999, Blood 93:3355-3368). The following primers were used for RT-PCR:

(SEQ ID NO:3)

| | |
|---|---|
| VEGFRI/Flt1 | 5′-AAACCAAGAC TAGATAGCGT CA-3′and |

(SEQ ID NO:4)

5′-TTCTCACATA ATCGGGGTTC TT-3′;

(SEQ ID NO:5)

| | |
|---|---|
| VEGFRII/Flt4 | 5′-GACAAGGAGT GTGACCACTG AA-3′and |

(SEQ ID NO:6)

5′-TGAAGGGACA TTGTGTGAGA AG-3′

(Klagsbrun et al., 1996, Cytokine Growth Factor Rev. 7:259-270).

The following primers, were also used:

(SEQ ID NO:7)

| | |
|---|---|
| Tiel | 5′-GAGTCCTTCT TTGGGAGATA GTGA-3′and |

(SEQ ID NO:8)

5′-GTCAGACTGG TCACAGGTTA GACA-3′;

(SEQ ID NO:9)

| | |
|---|---|
| Tek | 5′-CATTTTTGCA GAGAACAACA TAGG-3′and |

(SEQ ID NO:10)

5′-TCAAGCACTG GATAAATTGT AGGA-3′

(Sato et al., 1995, Nature 376:70-74).

CD34⁻lin⁻ cell purification

Purification of CD34⁻lin⁻ cells was performed as indicated in Bhatia et al. (Nature Med. 4:1038-1045). The KDR⁺ cell sub-fraction of the CD34⁻lin⁻ cell fraction was obtained as indicated herein for CD34⁺ cells.

The results of the Experiments presented herein are now described.

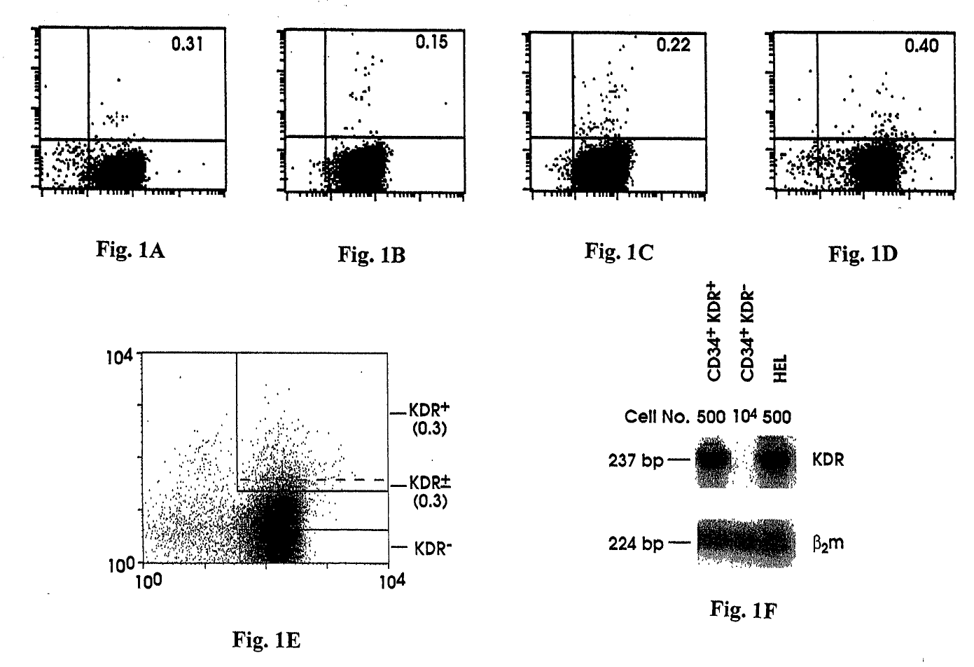

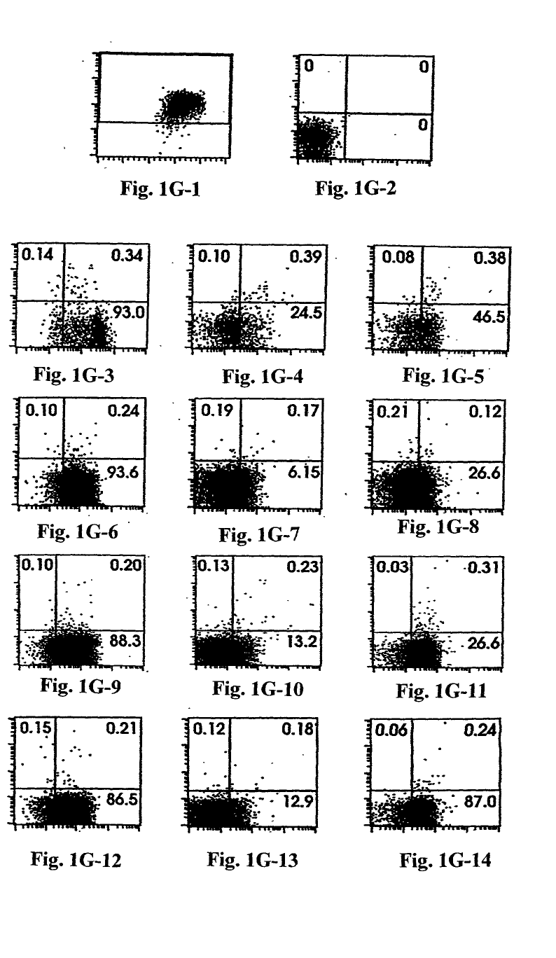

In preliminary studies, PB HPCs were purified and grown in unilineage differentiation cultures (Gabbianelli et al., 1990, Science 249:1561-1564; Testa et al., 1996, Blood 88:3391-3406). In accord with previous studies, RT-PCR analysis confirmed that KDR mRNA was expressed in HPCs, but was not detected in the HPC progeny except for expression on megakaryocytes (Katoh et al., 1995, Cancer Res. 55:5687-5692). Thereafter, a high-affinity monoclonal antibody (MoAb) which specifically binds the extracellular KDR domain was used to monitor KDR expression on HPCs from bone marrow (BM; FIG. 1A), normal peripheral blood (PB; FIG. 1B), mobilized peripheral blood (MPB; FIG. 1C), and cord blood (CB; FIG. 1D). Extensive FACS analysis on ≧98% purified CD34⁺ cell populations from these tissues indicated that KDR⁺ cells represent a minuscule subset of all CD34⁺ cells, usually comprised in the <1% range (FIGS. 1A through 1D) as confirmed by RT-PCR analysis (FIG. 1F). A KDR^{±} (KDR^{dim}) cell population has also been identified in CD34⁺ cells (FIG. 1E) and occasionally co-sorted with the KDR⁺ (KDR^{bright}) fraction. BM, PB, MPB, and CB CD34⁺KDR⁺ cells, essentially lin⁻(approximately <5-20% CD45RA⁺, CD13⁺, CD33⁺, CD61⁺, CD 19⁺ in representative experiments), are variably positive for early HPC/HSC markers (FIG. 1G).

The hematolymphopoietic hierarchy is defined by functional assays. Pluripotent HSCs, endowed with extensive self-renewal capacity, are assayed in vivo on the basis of their capacity to repopulate the hematolymphopoietic system, i.e., to xenograft irradiated NOD-SCID mice and pre-immune fetal sheep (Bhatia et al., 1997, Proc. Natl. Acad. Sci. USA 94:5320-5325; Wang et al., 1997, Blood 89:3919-3924; Conneally et al., 1997, Proc. Natl. Acad. Sci. USA 94:9836-9841; Zanjani et al., 1998, Exp. Hematol. 26:353-360). Primitive HPCs with limited self-renewal potential but extensive proliferative capacity, are identified in vitro as high proliferative potential colony-forming cells, HPP-CFCs (Brandt et al., 1990, J. Clin. Invest. 86:932-941). Lineage(s)-committed HPCs with no self-renewal activity (defined in vitro as colony-forming units {CFUs} or burst-forming units {BFUs}; Ogawa, 1993, Blood 81:2844-2853).

The 5-8 week LTC identifies LTC initiating cells (LTC-ICs), which represent primitive HPCs apparently distinct from in vivo repopulating HSCs (Larochelle et al., 1996, Nature Med. 2:1329-1337). The 12 week extended LTC identifies more primitive LTC-ICs, which are resistant to retroviral gene transfer, as repopulating HSCs, and represent putative HSCs (Hao et al., 1996, Blood 88:3306-3313; Larochelle et al., supra). Similarly, the LTC identifies 5-week and 12-week cobblestone area forming cells (CAFCs; van der Loo and Ploemacher, 1995, Blood 85:2598-2606). The data disclosed herein, utilizing the HSC/HPC functional assays, demonstrate that in post-natal hematopoietic tissues, KDR represents a specific functional HSC marker, which is virtually not expressed on oligo-unipotent HPCs.

In vitro HPC/HSC assays

CD34⁺KDR⁺ cells were tested by in vitro HPC/HSC assays. Preliminary studies indicated that VEGF addition in CD34⁺ cell culture exerts a mild stimulatory effect on multipotent CFU (CFU-Mix), HPP-CFCs, and 8 week LTC-ICs. Thereafter, CD34⁺ cells were purified and the CD34⁺KDR⁺ or CD34⁺KDR^{+/±} sub-fractions were separated from the CD34⁻KDR⁻ sub-fraction (FIG. 1E). Both subsets were then assayed for HPCs, HPP-CFCs and 6, 9, and 12 week CAFCs or 5, 8, and 12 week LTC-ICs.

HPC assay

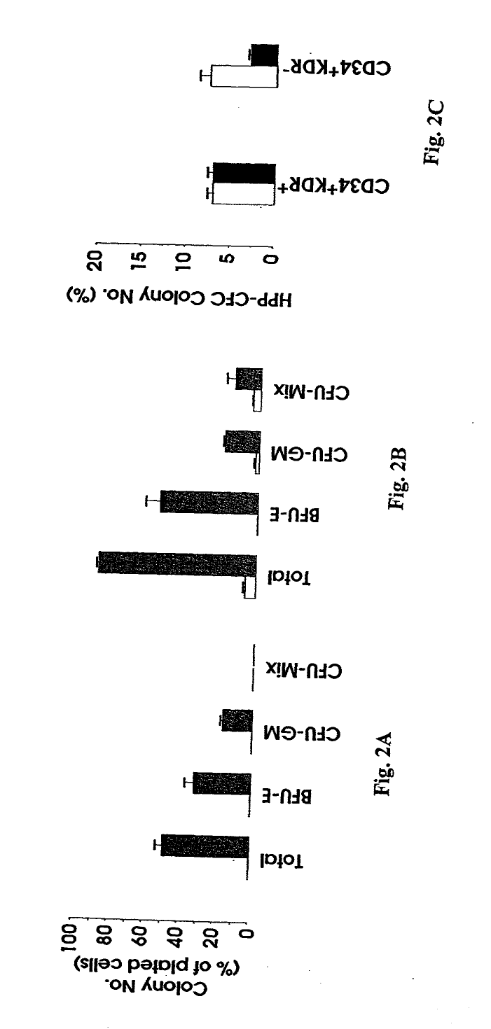

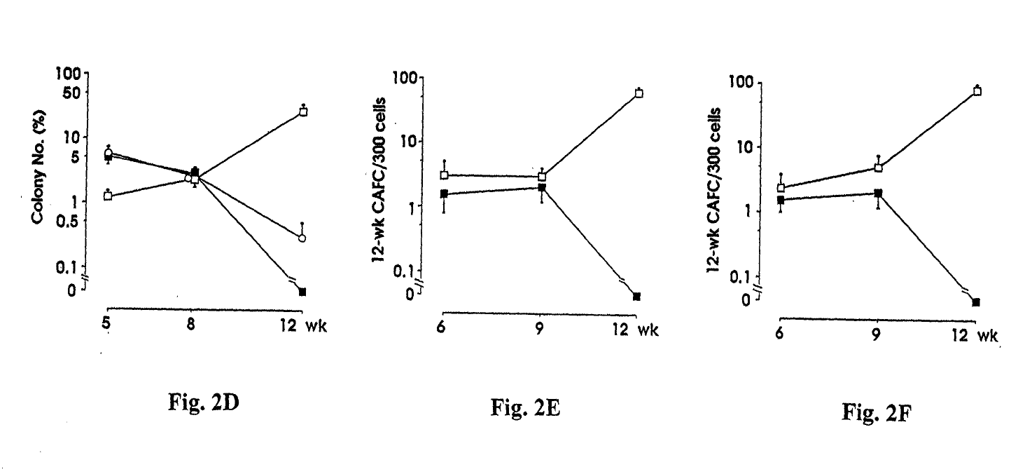

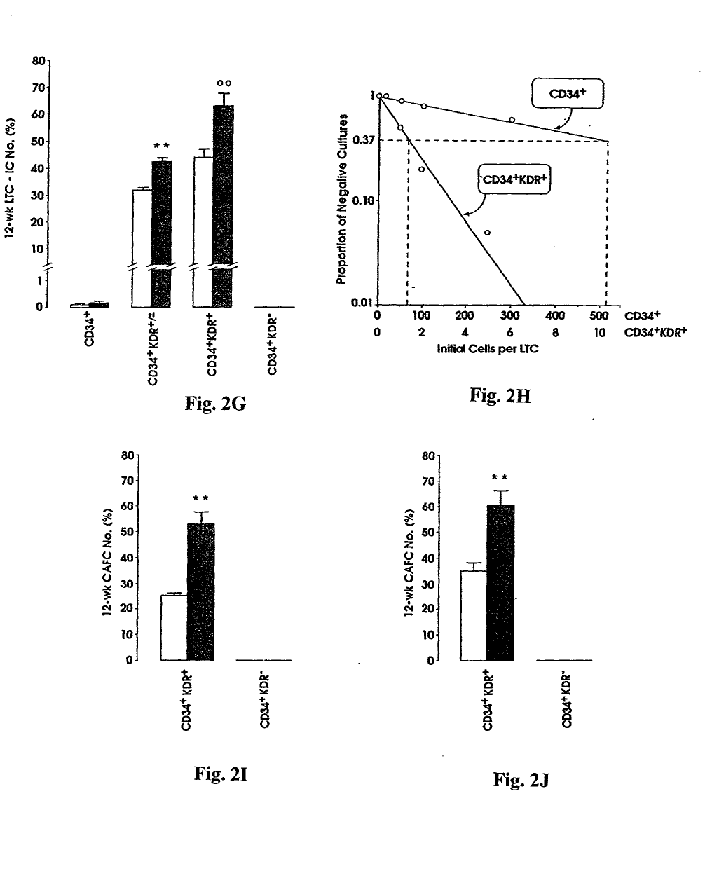

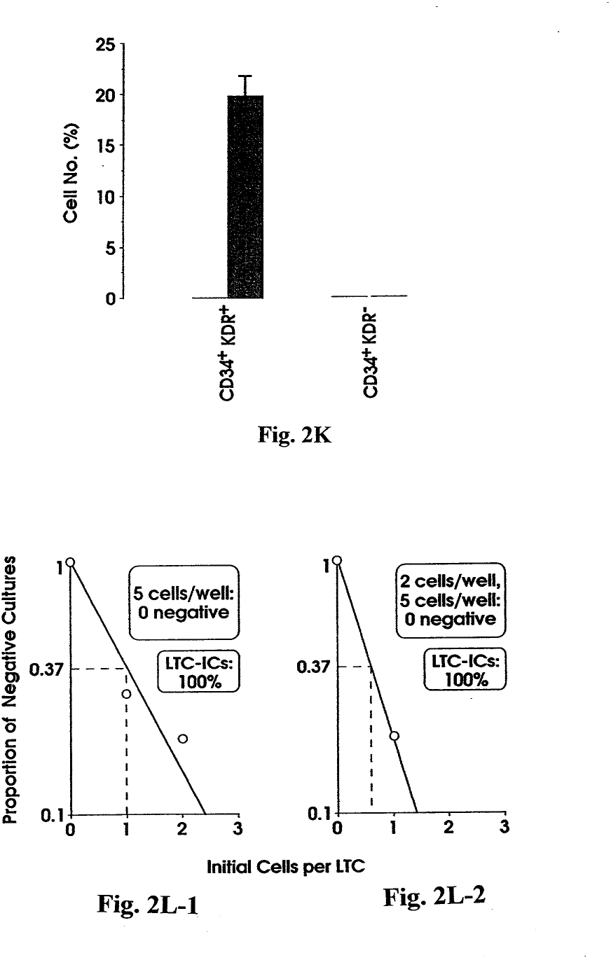

In representative PB experiments, the addition of saturating levels of interleukin 3 (IL3), granulomonocytic colony-stimulatory factor (GM-CSF) and erythropoietin (Epo) demonstrated that oligo-unipotent HPCs (BFU-E, CFU-GM) were essentially restricted to the KDR⁻ cell fraction (FIG. 2A). Addition of a larger spectrum of HGFs, i.e., including early-acting HGFs (c-kit ligand (KL), flt3 ligand (FL), and IL6) as well as unilineage HGFs (thrombopoietin (Tpo), G-CSF, and M-CSF), confirmed that virtually all oligo-unipotent HPCs are present in the KDR⁻ fraction (FIG. 2B). VEGF addition to the HGF cocktail did not modify this pattern, except for borderline increase of CFU-Mix in KDR⁺ culture. Essentially similar results were obtained for CB, MPB and PB.

HPP-CFC assay

HPP-CFCs scored in primary and secondary cultures (i.e., HPP-CFCs I and II, respectively) were present in both KDR⁺ and KDR⁻ fractions. The frequency of HPP-CFC II was more elevated in the KDR⁺ fraction (<10%) as compared to the KDR⁻ (<5%) population (results for PB are shown in FIG. 2C). Again, VEGF addition did not significantly modify this pattern, except for a slight increase of HPP-CFC number in the KDR⁺ cell culture. Similar results were obtained for BM and CB.

LTC-IC/CAFC assay

LTC-IC assay was performed in 5-, 8- and 12-week Dexter-type LTCs for CD34⁺, CD34⁺KDR^{+/±} and CD34⁺KDR⁻ cells from BM, MPB, PB, and CB (see, e.g., FIGS. 2D through 2F). The data disclosed in FIG. 2D demonstrate that in LTC seeded with PB CD34⁺ cells, the number of HPC generated declined sharply from 5 through 12 weeks, but a small residual number of HPCs was still detected at 12 weeks. In CD34⁺KDR⁻ LTC, a similar decline was observed, but no residual HPCs were detected at 12 weeks. Notably, CD34⁺KDR⁺ LTC exhibited a moderately low number of HPCs at 5 and 8 weeks, followed by a sharp increase of HPC generation at 12 weeks. An equivalent pattern was observed in BM (FIG. 2E), MPB and CB (FIG. 2F) LTC, as evaluated in 6, 9, and 12 week CAFC assay.

Altogether, oligo-unipotent HPCs are essentially restricted to KDR⁻ cells, while putative HSCs (12 week CAFCs/ LTC-ICs) are restricted to KDR⁺ cells. The intermediate primitive HPC populations (HPP-CFCs, 6-9 week CAFCs, 5-8 week LTC-ICs) are present in both cell fractions.

NOD-SCID mouse assays

Irradiated NOD-SCID mice were transplanted with CD34⁺ (50,0000 to 250,000 cells per mouse), CD34⁺KDR⁺ or CD34⁺KDR^{+/±} (150 to 10,000 cells per mouse), or CD34⁺KDR⁻ (10,000 to 250,000 cells per mouse) from BM, CB, MPB or PB. In some experiments, CD34⁻lin⁻KDR⁺ cells were also injected. Mice recipients were sacrificed at 12 weeks post-transplant and cell suspensions were obtained from BM, spleen and PB of mouse recipients and were analyzed by FACS for the presence of human cells as described herein. Consistent engraftment was observed using CD34⁺KDR⁺ cells and essentially no engraftment was observed using double sorted CD34⁺KDR⁻ donor cells.

NOD-SCID bone marrow studies

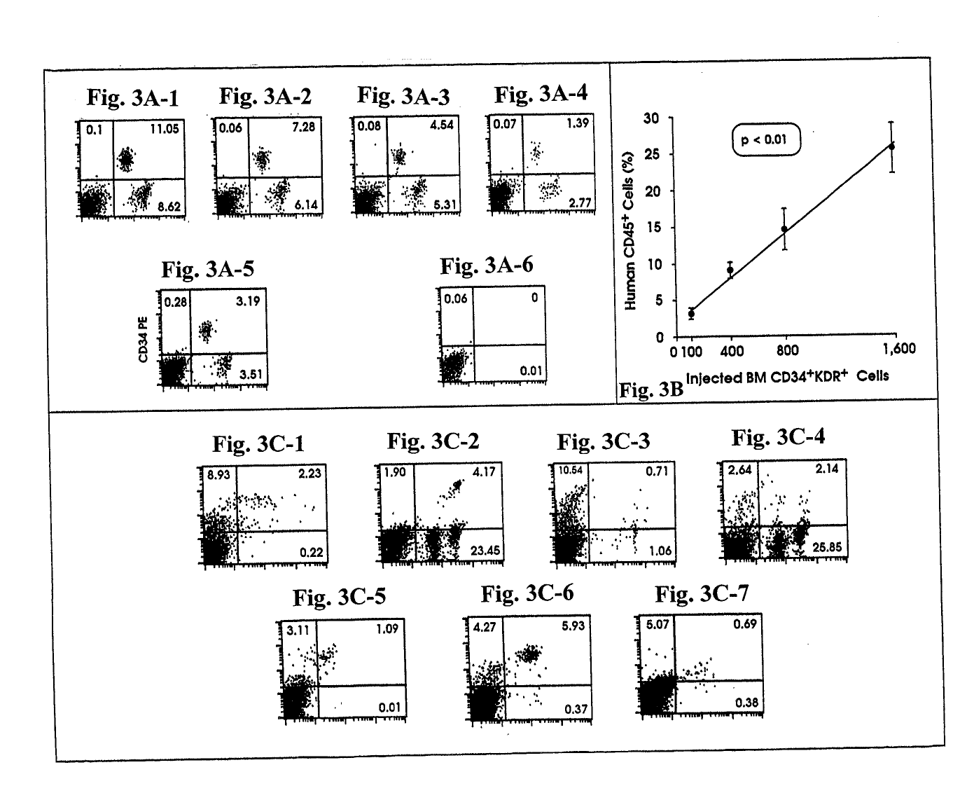

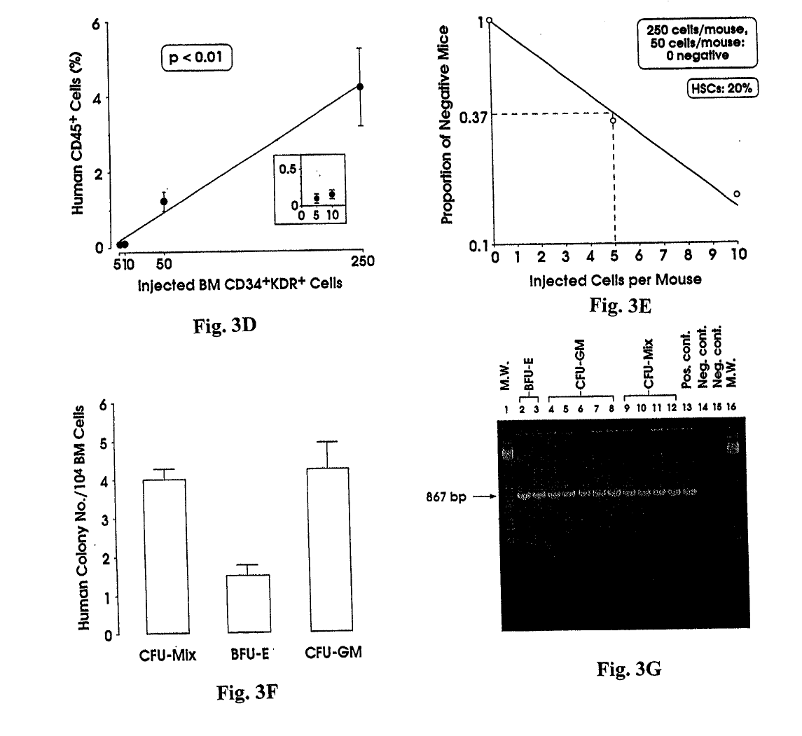

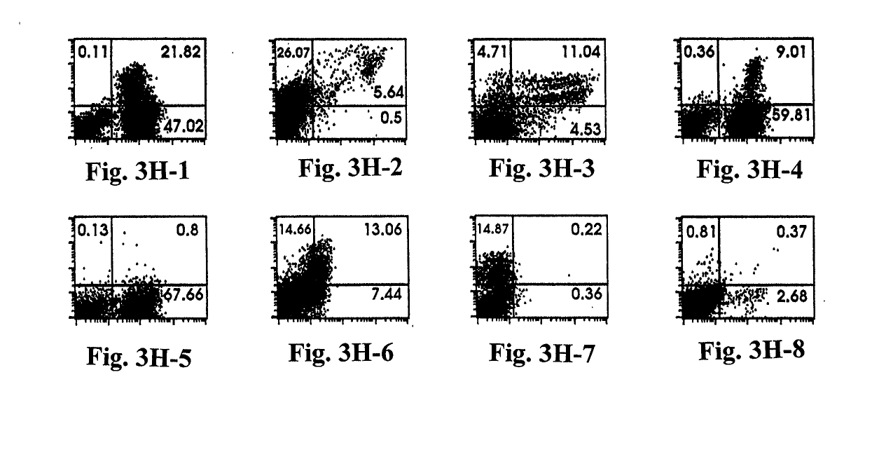

In a representative experiment (FIGS. 3A through 3C), between about 100 to about 1,600 CD34⁺KDR⁺ cells were injected into each NOD-SCID mouse recipient. In the negative control group, 250,000 double sorted CD34⁺KDR⁻ cells did not engraft, whereas unseparated CD34 cells demonstrated multilineage engraftment (FIG. 3A). CD34⁺KDR⁺ cells always engrafted the recipient mouse. Moreover, the engraftment observed involved all hematopoietic lineages (i.e., double labeling for CD33⁺15⁺or CD14⁺45⁺cells, CD71⁺GPA⁺cells, and CD45⁺41⁺ cells, pertaining to granulomonocytic, erythroid and megakaryocytic series, respectively) in representative mice (FIG. 3C). Further, the engraftment involved both B and T lymphoid compartments (i.e., CD19⁺20^{+and} CD4⁺8⁺3⁺cells, respectively), as well as NK cells (CD16⁺56⁺cells) (FIG. 3C). A dose-response was observed from 100 through 1,600 cells for all engrafted cell populations (FIG. 3B), particularly for CD45⁺cells (FIG. 3B). Although T cell precursors require specific cognate interaction for maturation, human CD34⁺CD4⁺CD8⁺and CD3⁺CD2⁺cells were generated in NOD-SCID mice BM following injection of CD34⁺CD38⁻cells or CD34⁻lin⁻ cells (Bhatia et al., 1997, Proc. Natl. Acad. Sci. USA 94:5320-5325; Verstegen et al., 1998, Blood 91:1966-1976; Bhatia et al., 1998, Nature Med. 4:1038-1045). Also, in vitro experiments in the prior art indicate that the BM microenvironment is permissive for T cell development, and can recapitulate thymic maturation (Garcia-Ojeda et al., 1998, J. Exp. Med. 187:1813-1823). Further, without wishing to be bound by theory, the presence of contaminant mature human T cells in the transplanted CD34⁺KDR⁺ cells can be excluded in view of the lack of human T lymphocytes in mice receiving large numbers of CD34⁺KDR⁻ cells. Thus, the data disclosed herein demonstrate that human T cell precursors develop in BM of NOD-SCID mice. Taken together, these data establish that the CD34⁺KDR⁺ population, but not the CD34⁺KDR⁻ subset, is capable of establishing long-term (3 month) human hematopoiesis of the various hematopoietic lineages in NOD-SCID mice recipients.

NOD-SCID cord blood studies

In five independent experiments, 200 to 15,000 CD34⁺KDR⁺ or 10,000 to 200,000 CD34⁺KDR⁻ CB cells were xenotransplanted into NOD-SCID mice. Human cells were virtually absent from mice transplanted with double sorted KDR⁻ cells. In contrast, KDR⁺ cells consistently generated human CD45⁺ cells in BM, PB, and spleen of the recipient mice according to a dose-dependent pattern, e.g., representative results indicate that mice receiving 1,000 to 10,000 cells exhibited 27.2±7.1% (mean±SEM) human CD45⁺ BM cells, whereas animals receiving 200 to about 800 cells demonstrated 3.75±1.5% CD45⁺ BM cells. In a representative experiment, mice transplanted with 6,000 CD34⁺KDR⁺ cells (FIG. 3H) exhibited abundant BM human CD34 progenitors, precursors of the erythroid, granulomonocytic, and megakaryocytic lineages, as well as B and NK cells. The low CD3 expression detected may, without wishing to be bound by theory, reflect the low T cell generation potential of CB HSCs.

Multilineage engraftment of sheep fetuses using CD34⁺KDR^{+/±} cells

BM studies involving CD34⁺KDR⁺ cells similar to those performed in NOD-SCID mice and disclosed herein were also performed in fetal sheep.

In a representative experiment, CD34⁺ cells were purified from two human BM samples. The CD34⁺KDR^{+/±} or the CD34⁺KDR⁻ sub-fraction was then injected into the pre-immune fetuses of eight pregnant ewes. The primary recipients received CD34⁺KDR^{+/±}, CD34⁺KDR⁻, or CD34⁺ cells (four, three, and two fetuses per group, respectively) and the recipients were then sacrificed on day 60 post-transplant. Other fetuses injected with CD34⁺KDR^{+/±} or with CD34⁺KDR⁻ cells were born. In addition, human CD34⁺ cells from primary fetuses treated with KDR^{+/±} cells were transplanted into secondary fetuses (Kawashima et al., 1996, Blood 88:4136-4142; Civin et al., 1996, Blood 88:4102-4109).

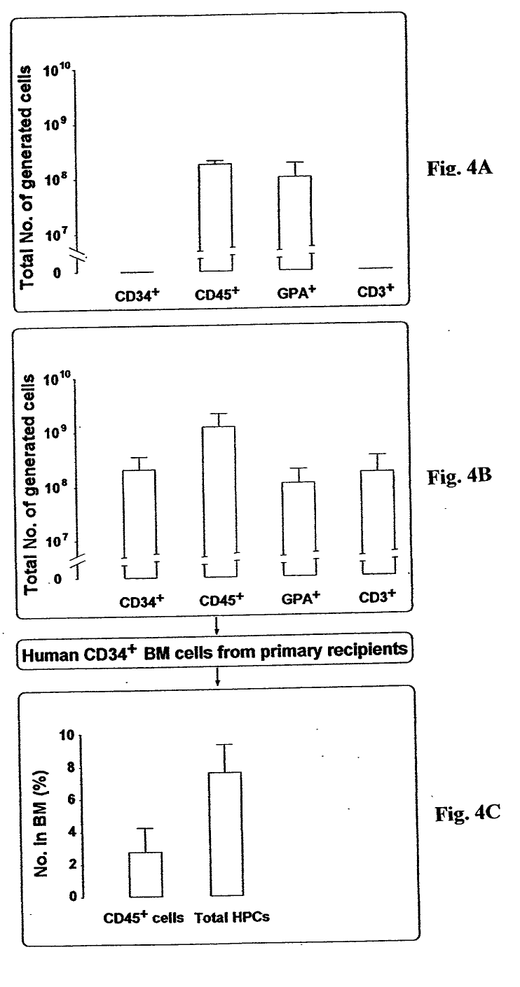

In primary fetal sheep recipients, transplantation of 1.2×10⁵ CD34⁺ cells per fetus consistently induced engraftment; that is, BM analysis indicated the presence of a significant fraction of differentiated (0.30% CD45⁺ cells, mean values) and undifferentiated (0.17% CD34⁺ cells) hematopoietic precursors. Further, clonogenic assay demonstrated that 6.8% CFU-Mix/BFU-E and 5.2% CFU-GM of all scored colonies were of human origin. A small number (3×10³ cells per fetus) of CD34⁺ KDR^{+/±} cells consistently engrafted with an impressive multilineage expression for the differentiated compartments: 1.78% CD45⁺, 0.16% GPA⁺, and 0.34% CD3⁺ cells. Further, these fetuses exhibited a consistent engraftment with multilineage expression for the undifferentiated compartment: 0.32% CD34⁺. Within the HPC pool, the frequency of human HPCs was elevated, i.e., 9.3% for CFU-Mix/BFU-E and 16.2% for CFU-GM of all scored colonies were of human origin. An 80-fold larger number (2.4×10⁵ cells per fetus) of CD34⁺KDR⁻ cells did not engraft any fetus, as indicated by the consistent absence of CD34⁺ and CD3⁺ cells. Moreover, only a small percentage of differentiated hematopoietic precursors was detected (i.e., 0.7% CD45⁺ cells), together with a few late CFU-GM (2.4%) giving rise to small colonies. It is estimated that more than 10⁸ CD34⁺ and CD3⁺ human cells were generated per fetus by KDR⁺ cells, whereas no CD34⁺ and CD3⁺ cells were generated by KDR⁻ cells (FIGS. 4A and 4B).

Each secondary fetal sheep recipient received 4×10⁵ human BM CD34⁺ cells, derived from the primary fetuses originally transplanted with KDR^{+/±} cells. After two months, the four secondary recipients were sacrificed and all demonstrated multi-lineage engraftment (FIG. 4C).

In born sheep recipients at three weeks after birth, both sheep transplanted with KDR cells in fetal life exhibited persistent multilineage engraftment at the BM level. One sheep featured an extremely abundant progeny of human CD45⁺ cells and 8.8% colonies of human origin, and the other sheep exhibited 1.0% CD45⁺ cells (the colony number was not evaluated for this sheep due to bacterial contamination of the culture plates).

These representative fetal sheep results, confirmed in other experiments, indicate that the CD34⁺KDR^{+/±} fraction is enriched for HSCs giving rise to multilineage engraftment in primary/secondary fetuses and born sheep. The engraftment in secondary recipients is noteworthy. Indeed, positive results in secondary fetal recipients successfully compare with those observed by follow up to primary transplanted fetuses for long periods after birth (Civin, 1996, Blood 88:4102-4109). On the other hand, the CD34⁺KDR⁻ fraction does not engraft and contains only HPCs giving rise, in primary recipients, to differentiated hematopoietic precursors and a few late CFU-GM.

In sum, the data disclosed herein regarding the NOD-SCID and fetal sheep xenotransplantation assays indicate that restriction of HSCs to the KDR⁺ sub-fraction of CD34⁺ cells. Previous studies in NOD-SCID mice and in sheep fetuses demonstrated that HSCs are enriched in diverse CD34⁺ cell sub-fractions, e.g., CD38⁻, kit^{low}, Thy-1⁺, and Rhodamine (Rh)^{dim} (Bhatia et al., 1997, Proc. Natl. Acad. Sci. USA 94:5320-5325; Wang et al., 1997, Blood 89:3919-3924; Conneally et al., 1997, Proc. Natl. Acad. Sci. USA 94:9836-9841; Verstegen et al., 1998, Blood 91:1966-1976; Civin et al., 1996, Blood 88:4102-4109; Kawashima et al., 1996, Blood 87:4136-4142; Sutherland et al., 1996, Exp. Hematol. 24:795-806; Uchida et al., 1996, Blood 88:1297-1305). However, engraftment was also observed at a lower level for the complementing sub-fractions, i.e., CD38⁺, kit⁻, Thy-1⁻, and Rh^{bright} (Conneally et al., 1997, Proc. Natl. Acad. Sci. USA 94:9836-9841; Verstegen et al., 1998, Blood 91:1966-1976; Civin et al., 1996, Blood 88:4102-4109; Kawashima et al., 1996, Blood 87:4136-4142; Sutherland et al., 1996, Exp. Hematol. 24:795-806; Uchida et al., 1996, Blood 88:1297-1305).

Frequency of repopulating HSCs and 12-week CAFCs/LTC-ICs in CD34⁺KDR⁺ cell fraction

In NOD-SCID mice injected with from about 100 to about 1,600 BM CD34⁺KDR⁺ cells, the representative CD45⁺ cell dose-response (FIG. 3B) indicated that a cell number far lower than 100 cells would successfully engraft. Therefore, a representative LDA was performed using 250, 50, 10 or 5 BM CD34⁺KDR⁺ cells per mouse (FIG. 3D). After injection of 250 to 5 BM KDR⁺ cells, a dose-dependent multilineage engraftment was detected (FIGS. 3D and 3F). All mice were repopulated by 250 and 50 cells, while five of six mice injected with 10 cells and four of 6 mice injected with 5 cells were engrafted based on flow cytometry analysis (FIG. 3D) and HPC assay validated by PCR of human alpha-satellite DNA in the scored colonies (FIG. 3G). LDA indicated an approximately 20% frequency value for repopulating HSCs in CD34⁺KDR⁺ cells (FIG. 3E). This representative value is similar to the representative 25% CAFC frequency exhibited in VEGF⁻BM LTC, indicating that repopulating HSCs and 12 week LTC-ICs/CAFCs are closely related.

In representative experiments on 12 week extended LTCs treated or not treated with VEGF, LDA indicated that the CAFC frequency in CD34⁺KDR⁺ cell of BM (FIG. 21) or CB (FIG. 2J) CAFC is lower in VEGF⁻ (approximately 25-35%) than in VEGF⁺ (approximately 53-61%) LTC. No CAFC were detected in CD34⁺KDR⁻ cell fractions.

Representative corresponding experiments on LTC-IC frequency in CD34⁺KDR⁻ or CD34⁺KDR⁺ fractions from BM, CB, MPB and PB showed a pattern similar to that observed for CAFC frequency.

The 20% repopulating HSCs frequency in CD34⁺KDR⁺ BM cells was about 100-fold more elevated than the frequency reported in CD34⁺CD38⁻ BM or CB cells (Bhatia et al., 1997, Proc. Natl. Acad. Sci. USA 94:5320-5325; Wang et al., 1997, Blood 89:3919-3924; Conneally et al., 1997, Proc. Natl. Acad. Sci. USA 94:9836-9841). It is noteworthy that in representative experiments the CD34⁺CD38⁻ fraction comprises about <10% KDR⁺ cells. This result explains the different HSC frequency in the CD34⁺38⁻ subset compared to the frequency in the CD34⁺KDR⁺ cell subset. The assay performed herein lasted for 3 months and the mice were not treated with cytokines, whereas in other studies the assay usually lasts 1.5 to 2 months and often involves cytokine treatment (Larochelle et al., 1996, Nature Med. 2:1329-1337).

Representative in vitro LDAs indicated that 25 to 35% of CAFCs were present in BM and CB CD34⁺KDR⁺ cells, as evaluated in VEGF⁻ 12-week LTC. Without wishing to be bound by theory, since the CAFC frequency rises to 53 to 63% in these representative VEGF⁺ LTCs, it is predicted that the in vivo repopulating HSC frequency will be more elevated in mice injected with human VEGF i with or without other cytokines. Importantly, the significant increase of CAFC/LTC-IC frequency induced by VEGF addition suggests that VEGF exerts a key proliferative and/or anti-apoptotic effect on putative HSCs.

Increased 12 week CAFC/LTC-IC frequency in starvation-resistant CD34⁺KDR⁺ cells

The 12-week LTC-IC frequency in starvation-resistant CD34⁺KDR⁺ or CD34⁺KDR^{+/±} cells was examined. In representative experiments, CD34⁺KDR⁺ or CD34⁺KDR^{+/±} and CD34⁺KDR⁻ cells were seeded into FCS⁻ free liquid suspension minibulk cultures, supplemented with VEGF but deprived of other HGFs. The KDR⁺ or KDR^{+/±} cell number decreased sharply in the first five days of culture, but then leveled down to 10-25% residual cells through day 30. Conversely, all KDR⁻ cells were dead at day 10 of culture. In single CD34⁺KDR⁺ cell starvation cultures not supplemented by VEGF all cells died while approximately 20% of cells treated with VEGF survived (FIG. 2K), indicating the key anti-apoptotic effect of VEGF on this cell type.

The starvation-resistant KDR^{+/±} fraction contained virtually no multipotent/primitive HPCs (as determined by CFU-Mix/HPP-CFC assays), but exhibited an elevated 12 week LTC-IC frequency, approximately ≧80-95% at day 5-30 (FIG. 2L). Control KDR⁻ cells never contained 12 week LTC-ICs. Without wishing to be bound by theory, based on the similarity between in vivo and in vitro HSC assay results, it may be that the starvation-resistant CD34⁺KDR⁺ cells represent HSCs having in vivo long-term repopulating capacity. The data disclosed herein are in accord with prior studies demonstrating that one of the key features of adult HSCs is their quiescent status in a prolonged cell cycle (Ogawa, 1993, Blood 81:2844-2853; Morrison et al., 1997, Cell 88:287-298; Orlic and Bodine, 1994, Blood 84:3991-3994). That is, the high frequency of HSCs in CD34⁺KDR⁺ cells capable of withstanding serum starvation may be due to their ability to remain quiescent which is a known characteristic of adult HSCs thus further suggesting that KDR⁺ is a marker specific for HSCs.

HSCs in CD34⁻/lin⁻/KDR⁺ cells

Experimental and clinical observations leave little doubt that human HSCs with long-term engrafting ability are CD34⁺ (Berenson et al., J. Clin. Invest. 81:951-955; Berenson et al., 1991, Blood 77:1717-1722; Bensinger et al., 1996, Blood 88:4132-4138). This has also been confirmed not only in the SCID mouse models, but also in the sheep models where CD34⁺ cells have caused engraftment lasting>5 years (Zanjani et al., 1996, Int. J. Hematol. 63:179-192). However, recent studies in both mice and rhesus monkeys have demonstrated the CD34⁻ cells population contain progenitors capable of producing CD34⁺ cells in vitro and to be highly enriched in HSCs with competitive long-term in vivo repopulating potential (Osawa et al., 1996, Science 273:242-245; Goodell et al., 1996, J. Exp. Med. 183:1797-1806; Johnson et al., 1996, Blood 88:629a).

Recent reports suggest that in the sheep fetus large numbers (>10⁵) of human BM CD34⁻ cells can engraft (Zanjani et al., 1998, Exp. Hematol. 26:353-360; Almeida-Porada et al., 1998, Exp. Hematol. 26:749).

Furthermore, studies by Bhatia et al. (1998, Nature Med.4:1038-1045) indicate that 1-2×10⁵ BM or CB CD34⁻lin⁻ cells engraft a majority of NOD-SCID mice after 2-3 months, with generation of CD34⁺ cells and multilineage expression including B and T lymphocytes. The data disclosed herein demonstrate that NOD-SCID mice injected with 4,000 CD34⁻lin⁻KDR⁺ CB cells consistently exhibited CD34⁺ cell generation and multilineage engraftment after three months. Specifically, the following representative values were detected in BM: 0.19% CD34⁺ and 0.11% CD34⁺CD45⁺cells, coupled with multilineage expression (e.g., 0.23% CD45⁺, 0.18% CD33⁺, 0.10% CD15⁺, 0.27% GPA⁺, 0.27% CD71⁺, 0.15% CD20^{+,} 0.12% CD19⁺, 0.25% CD3⁺, and 0.11% CD56⁺CD16⁺). In the same experiment, 4,000 CD34⁻KDR⁺ cells engrafted. Furthermore, 10,000 KDR⁺ CB mononuclear cells engrafted, whereas 100,000 KDR⁻ CB mononuclear cells did not engraft.

A large number of human BM and CB CD34⁻lin⁻ cells engraft fetal sheep and NOD-SCID mice, as indicated by multilineage expression and generation of a CD34⁺ cells. Approximately one percent or less of CD34⁻/lin⁻ cells are KDR⁺. Indeed, a discrete number of CB CD34⁻lin⁻KDR⁺ cells engraft NOD-SCID mice and generate CD34⁺ cells. Based on these results, and without wishing to be bound by theory, KDR is a key marker for CD34⁻ HSC in post-natal life.

Although HSCs have previously been enriched in diverse CD34⁺ cell subsets, a HSC defining marker had not, prior to the present invention, been identified. The data disclosed herein demonstrate that the CD34⁺KDR⁺ cell fraction has novel properties. HSCs are essentially restricted to this population, whereas oligo-unipotent HPCs are virtually restricted to CD34⁺KDR⁻ cells. Further, the HSC enrichment in CD34⁺KDR⁺ cells is strikingly elevated, i.e., the putative HSC frequency rises to≧80-95% in starvation-resistant CD34⁺KDR⁺ cells. Altogether, these results indicate that KDR is a novel functional marker defining HSCs.

Purification of CD34⁺ HPCs has markedly facilitated studies on early hematopoietic precursors (Ogawa et al., 1993, Blood 81:2844-2853; Gabbianelli et al., 1990, Science 249:1561-1564). The isolation of KDR⁺ HSCs offers a unique opportunity to elucidate the cellular/molecular phenotype and functional properties of HSCs/HSC subsets. These issues, exceedingly elusive so far, are of pivotal significance for a large array of biotechnological and clinical aspects, e.g., autologous/allogeneic HSC transplantation, in vitro blood cell generation for transfusion medicine, and HSC gene therapy in hereditary/acquired hematology-immunology disorders.

The data disclosed herein shed light on recent studies on embryonic hematoangiogenesis. Studies on Flk-1^{-/-} knock out mice indicate that Flk-1 is required to initiate both primitive and definitive hematolymphopoiesis, as well as vasculogenesis (Shalaby et al., 1997, Cell 89:981-990). These data suggest a role for Flk-1 in generation of hemoangioblasts, i.e., putative stem cells for both hematolymphopoietic and endothelial lineages (Flamme et al., 1992, Development 116:435-439). Flk-1⁺ and CD34⁺ cells are present in murine embryonic-fetal liver (Kabrun et al., 1997, Development 124:2039-2048). In differentiating embryonic stem cells, embryoid bodies treated with VEGF and KL give rise to CD34⁺ and flk-1⁺ blast cell colonies, which generate secondary colonies composed of all hematopoietic lineages and which also exhibit endothelial developmental capacity (Kennedy et al., 1997, Nature 386:488-492; Nishikawa et al., 1998, Development 125:1747-1757; Choi et al., 1998, Development 125:725-732).

Altogether, previous studies suggested the existence of embryonic CD34⁺flk-1⁺ hemoangioblast, but did not provide evidence for a prenatal CD34⁺flk-1⁺ repopulating HSC. The data disclosed herein demonstrate the existence of post-natal CD34⁺KDR⁺ repopulating HSC. Furthermore, the data disclosed herein demonstrate the existence of post-natal CD34⁺KDR⁺ hemoangioblasts. Without wishing to be bound by theory, taking together the data disclosed herein, KDR-flk-1 may hypothetically define both post-natal and pre-natal HSCs/hemoangioblasts.

Recently, bone marrow-derived cells have been demonstrated to give rise to hepatic oval cells, which can differentiate into the other two types of epithelial cells in the liver, i.e., ductular cells and hepatocytes (Petersen et al., 1999, Science 284:1168-1170). In addition, bone marrow-derived cells have been demonstrated to have the capability to give rise to myogenic progenitors (Ferrari et al., 1998, Science 279:1528-1530). Also, bone marrow-derived cells were induced to differentiate into the adipocytic, chondrocytic, or osteocytic lineages (Pittenger et al., 1999, Science 284:143-147). In mice, adult bone marrow cells that were transplanted intraperitoneally migrated into the brain of the recipient and differentiated therein to form cells that expressed neuron-specific antigens (Mezey et al., 2000, Science 290:1779-1782). Transplantation of murine bone marrow cells into another mouse led to generation of both microglia and macroglia in the brain of the recipient mouse (Eglitis et al., 1997, Proc. Natl. Acad. Sci. USA 94:4080-4085). Furthermore, murine HSC that were transplanted into primary recipients and subsequently re-transplanted into secondary recipients led to generation in the secondary recipients of hematopoietic cells and of epithelial cells of the liver, lung, gastrointestinal tract, and skin. Without wishing to be bound by any particular theory of operation, it is believed that the stem cells that gave rise to mesenchymal cells, hepatic oval cells, myogenic cells, neuronal cells, glial cells, and lung, gastrointestinal, and skin epithelial cells were KDR⁺ stem cells (e.g., it is believed that the cells that differentiated in Krauser et al., 2001, Cell 105:369-377 were KDR⁺ stem cells). Thus, the present invention provides methods of isolating and purifying cells which not only give rise to multilineage hematopoietic engraftment, but can also provide methods of targeting gene therapies to a wide variety of tissues including all of those described herein. Therefore, the prior art has only suggested that such multipotent cells existed, however, the present invention teaches how to obtain them, as demonstrated in the ensuing examples.

In summary, the major hurdle in studies on hematolymphopoietic stem cells (HSCs) has been the lack of an HSC-specific marker. The lack of a specific HSC marker hampered purification, characterization and utilization of this extremely rare cell population. The data disclosed herein demonstrate, for the first time, that the vascular endothelial growth factor receptor 2 (VEGFR2, KDR/Flk-1) is a specific functional marker for human HSCs in adult bone marrow (BM), normal or mobilized peripheral blood (PB, MPB), and cord blood (CB). In these post-natal tissues, pluripotent repopulating HSCs are virtually restricted to and highly purified in the miniscule CD34⁺KDR⁺ cell fraction (<1% of CD34⁺ cells), as evaluated in NOD-SCID mice and fetal sheep xenografts. This CD34⁺KDR⁺ cell fraction contains essentially no oligo-unipotent hematopoietic progenitor cells (HPCs). Conversely, oligo-unipotent HPCs are virtually restricted to and highly purified in CD34⁺KDR⁻ cells, which contain essentially no HSCs.

In a representative experiment, the frequency of repopulating HSCs in the BM CD34⁺KDR⁺ subset, evaluated in NOD-SCID mice by limiting dilution assay (LDA), is 20%; similarly, representative experiments showed that the frequency of putative HSCs (CAFC) in the BM CD34⁺KDR⁺ subset, evaluated by LDA in 12-week extended Dexter-type long term culture (LTC), was 25%. The frequency rose in LTC supplemented with VEGF (to 53% in representative experiments), thus suggesting a functional role for the VEGF/KDR system in HSCs. Conversely, putative HSCs were essentially not detected in the CD34⁺KDR⁻ subset. In addition, the fraction of CD34⁺KDR⁺ cells resistant to prolonged GF starvation (except for VEGF addition) in FCS-free culture comprises a very elevated frequency of putative HSCs,≧80-95% in representative experiments.

The data disclosed herein indicate that KDR is a functional HSC defining marker, which distinguishes HSCs from oligo-unipotent HPCs. The present invention makes possible the characterization and functional manipulation of HSCs/HSC subsets, as well development of innovative approaches for HSC clinical utilization. The data disclosed herein that KDR is a functional marker of primitive human stem cells, regardless of the source or tissue from which the stem cells are derived. Thus, although hematopoietic tissues are a relatively rich source of stem cells, expression of the KDR marker (i.e., exhibition of reactivity with an anti-KDR antibody) is an indication that a stem cell is primitive and exhibits a plasticity such that it can differentiate to form a cell of a large variety of tissues, regardless of the identity of the tissue from which the stem cell was isolated. Thus, all KDR⁺ stem cells (i.e., not just the KDR⁺ HSCs disclosed herein) can be used in the same manner as described herein for use of KDR⁺ HSCs. By way of example, KDR⁺ stem cells isolated from muscle tissue can be maintained in the presence of damaged neuronal tissue (e.g., in a stroke-lesioned area of a human brain) in order to induce differentiation of the stem cells into neuronal and other neural cells, thereby repairing at least some of the stroke-induced neural damage and alleviating the effects of the stroke.

### Example 2

Post-natal CD34⁺KDR⁺ Cells Generate Both Hematopoietic and Endothelial Cells in Minibulk Culture

In post-natal life, the CD34⁺KDR⁺ cell subset ( 1.5% of the whole CD34⁺ population) exhibits HSC activity, and contains endothelial precursors (Ziegler et al., 1999, Science 285:1553-1558; Peichev et al., 2000, Blood 95:952-958). The experiments presented in this example were designed to test the capacity of cord blood CD34⁺KDR⁺ cells to generate hematopoietic and endothelial progeny in serum-free liquid suspension cultures. A total of 36 experiments were performed.

CD34⁺KDR⁺ cells were sorted using KDR1/KDR2 MoAbs and were seeded in culture wells (-2000-4000 cells per 0.2 milliliter) in media supplemented with VEGF at saturating level. Control cultures were seeded with CD34⁻KDR⁻ cells. In all experiments we observed that, after 1-2 weeks, all CD34⁺KDR⁻ cells were dead. In contrast, 30-70% of CD34⁺KDR⁺ cells survived (this residual population, composed of small blast cells, is highly enriched for 12-week long-term culture initiating cells; Ziegler et al., 1999, supra). At later culture times, the blast cell population persisted and gradually generated a progeny of larger cells for up to 6 months. The cells were analyzed at sequential culture times by morphology, immunofluorescence, immunohistochemistry and RT_PCR analysis. The small blasts were CD45^{dim} or CD45⁻, while negative for markers of differentiated hematopoietic and endothelial cells (particularly, CD14 and Von Willebrand factor/vascular endothelial-cadherin; VW/VE-cadherin). Larger cells comprised three cell types: (a) monocytic/dendritic cells (CD45⁺CD14⁺, VW⁻) at different stages of differentiation; (b) endothelial cells (CD45⁻CD14⁻, VW⁺/VE-cadherin⁺) at sequential stages of development (from small mononucleated to large polynucleated cells); (c) a few, relatively small cells expressing both hematopoietic and endothelial markers, indicative that these cell are bipotent for both lineages. The experiments presented in this example indicate that the CD34⁺KDR⁺ cell population comprises both hematopoietic and endothelial precursors, thus in line with similar results obtained from in vitro differentiation of Flk1⁺ cells from adult murine bone marrow (Huang et al., 1999, Biochem. Biophys. Res. Comm.). Furthermore, these data demonstrate that few cells are bipotent for both lineages.

### Example 3

Identification of Hemoangioblasts in Post-natal CD34⁺KDR⁺ Cells

Post-natal CD34⁺ cells expressing KDR (VEGFRII, termed Flk1 in mice) generate hematopoietic or endothelial progeny or both in different in vitro/in vivo assays (Ziegler et al., 1999, Science 285:1553-1558; Peichev et al., 2000, Blood 95:952-958; Huang et al., 1999, Biochem. Biophys. Res. Comm. 264:133-138). The experiments presented in this example were designed to determine if human CD34⁺KDR⁺ cells comprise hemoangioblasts, i.e., stem/progenitor cells bipotent for both lineages. Minibulk culture of CD34⁺/KDR⁺ cells resulted in the generation of not only endothelial and hematopoietic progeny, but also a few cells co-expressing markers of both cell types. Therefore, a series of single cell culture experiments was performed on CD34⁺KDR⁺ cells separated by FACSVantage® utilizing KDR2 MoAb. Single cells were seeded in clonogenic culture (i.e., limiting dilution down to 0.25 cell per well) in standard collagen-/fibronectin-coated wells supplemented with fetal calf and horse sera (15% each), hematopoietic growth factors (GFs), and endothelial GFs. Specifically, the hematopoietic GFs were those used for the HPP-CFC assay disclosed herein. The endothelial GFs were basic fibroblast growth factor and VEGF, which also stimulate primitive hematopoietic cells, as indicated herein. In these culture conditions, CD34⁺KDR⁺ cells give rise to not only pure hematopoietic or endothelial colonies (<10% to 15% of plated cell number), but also mixed hematoendothelial clones (≦5% of plated cell number). Identification of hematopoietic and endothelial cells was based on immunofluorescence, immunohistochemistry, and RT-PCR assays designed to detect hematopoietic markers (e.g., CD45/CD14/CD41) and endothelial markers (e.g., VW factor, VE-cadherin, embryonic stem cell marker 2 (ESCM2), and TEK). For example, endothelial cells were CD45⁻CD14⁻CD41⁻, while positive for the endothelial markers. The results of these studies demonstrate that ≦5% of CD34⁺KDR⁺ cells are hemoangioblasts generating mixed hematoendothelial progeny.

Single CD34⁺KDR⁺ cells were seeded in Dexter-type long-term culture extended for 3 months (ELTC). Blast cells generated in a unicellular well were re-seeded for a second and then a third round of single cell ELTC. A minority (<5%) of blasts generated in tertiary ELTC gave rise in semisolid medium to mixed macroscopic colonies, composed of both hematopoietic and endothelial progeny, identified as indicated above. These observations indicated the capacity of CD34⁺KDR⁺ hemoangioblasts for extensive self-renewal.

CD34⁺KDR⁻ cells transduced with the Flk1 gene acquire the capacity to generate mixed hematoendothelial colonies in semisolid medium. Altogether, the experiments presented in this example identify post-natal hemoangioblasts in a CD34⁺KDR⁺ cell subset, endowed with long term proliferative potential and bipotent differentiation capacity. These bemoangioblasts may represent a lifetime reservoir/source of primitive hematopoietic and endothelial precursors, particularly precursors present in a CD34⁺KDR⁺ cell population. These observations further indicate that hemoangioblasts tested in assays permissive for either hematopoietic or endothelial differentiation can function as unipotent hematopoietic or endothelial stem cells, respectively. Example 4

Post-natal CD34⁺KDR⁺ Cells Injected into Murine Blastocyst Exhibit Multi-Tissue Plasticity in Mouse Embryos and Newborns

The experiments presented in this example were performed to test whether CD34⁺KDR⁺ cells possess a broad spectrum of differentiation potential. Human cord blood CD34⁺KDR⁺ cells and CD34⁺KDR⁻ HPCs were injected in non-immunocompromised murine blastocysts. The fate of the injected human cells during murine embryogenesis and post-natal life was followed. Human donor contribution was evaluated by chromosome 17-specific PCR on genomic DNA prepared from isolated embryonic and newborn tissues, as well as by immunohistochemistry analysis using standard tissue and human specific antibodies (e.g., human anti-albumin antibody for detection of human hepatocytes). Analysis was performed on a total of 74 embryos and newborns.

Human donor cells were detected in embryonic tissues isolated at 13.5 days of gestation as well as newborn tissues. The degree of chimerism was markedly more pronounced after injection of CD34⁺KDR⁺ cells, as compared to injection of CD34⁺KDR⁻ cells. The number of human cells in chimeric embryonic or chimeric newborn tissues was observed in a range of 5-20 human cells per 10⁵ murine cells and reached numbers as high as 500-1000 human cells per 10⁵ murine cells. In embryos the highest frequency of human cells was found in hematopoietic tissues. Human donor contribution persists in newborn mice injected with CD34⁺KDR⁺ cells; these mice exhibit human/mouse chimerism in multiple tissues, particularly in tissue of the central nervous system (brain and spinal cord), as well as in tissues of endo- or mesodermic origin (e.g., liver, lung, gut, skeletal muscle, heart, and kidney). The results obtained from the experiments presented in this example indicate that post-natal CD34⁺KDR⁺ cells not only comprise HSCs but also comprise cells with a capacity to differentiate into a variety of tissues of ecto-, meso-, and endodermic origin. Specifically, injection of human CD34⁺KDR⁺ HSCs into a murine blastocyst gives rise to a human-mouse chimeric brain and heart in the early embryonic/early post-natal life. Conversely, human CD34⁺KDR⁻ progenitor cells have little or no chimeric potential, and do not lead to formation of chimeric brain or heart following their injection into a murine blastocyst.

Example 5

Post-natal CD34⁺KDR⁺ Cells Generate Human Skeletal Muscle Cells in Regenerating Murine Muscle

The experiments presented in this example were performed to investigate the plasticity potential of CD34⁺KDR⁺ cells. CD34⁺KDR⁺ cells, purified from cord blood and adult peripheral blood, were analyzed for their capacity to differentiate into skeletal muscle cells in vivo. In other experiments, CD34⁻lin⁻KDR⁺ cells were analyzed for their differentiative capacity. Muscle damage was induced in SCID-Bg mice by standard cardiotoxin injection methods in the tibialis anterioris. Twenty-four hours later, between 100-10,000 cells CD34⁺KDR⁺ cells or between 100-10,000 CD34⁻lin⁻KDR⁺ cells were injected into the regenerating muscle. Duplicate samples were generated by treating both tibialis muscles in each mouse. At day 10 after cell injection, mice were sacrificed and tibialis muscles harvested. For each mouse, one tibialis muscle was analyzed by RT-PCR using human-specific primers for the muscle-specific master gene Myo-D. The second sample was snap frozen, cryosectioned, and analyzed by immunofluorescence (IF) and confocal microscopy for the presence of human nuclei (detected by an anti-human nuclei MoAb) interspersed within the muscle fibers. In four different experiments, as few as 100-10,000 CD34⁺KDR⁺ cells or CD34⁻lin⁻KDR⁺ cells provided a detectable signal by both RT-PCR and IF, indicating differentiation into skeletal muscle cells had occurred. Conversely, a 2-3 log higher number of CD34⁺KDR⁻ cells or CD34⁻lin⁻KDR⁻ cells generated no signal by either RT_PCR or IF. In summary, the data presented in this example indicate that the CD34⁺KDR⁺ or CD34⁻lin⁻KDR⁺ cell fraction from both peripheral blood and cord blood is endowed with the potential of differentiating into mesenchymal tissues other than that of origin, specifically, skeletal muscle cells.

The disclosures of each and every patent, application and publication cited herein are hereby incorporated herein by reference in their entirety.

While this invention has been disclosed with reference to specific embodiments, other embodiments and variations of this invention can be devised by others skilled in the art without departing from the true spirit and scope of the invention. The appended claims include all such embodiments and equivalent variations.

What is claimed is:

1. A method of generating a differentiated human cell of a selected type, the method comprising maintaining an isolated human KDR⁺ stem cell in the presence of a differentiated mammalian cell of the selected type, whereby the stem cell differentiates to become the differentiated human cell of the selected type.

2. The method of claim 1, wherein the stem cell is maintained in contact with the differentiated mammalian cell.

3. The method of claim 1, wherein the stem cell is maintained in vitro in the presence of the differentiated mammalian cell.

4. The method of claim 1, wherein the stem cell is separated from the differentiated mammalian cell by a porous barrier.

5. The method of claim 1, wherein the stem cell is isolated from a human hematopoietic tissue using a reagent that specifically binds with KDR.

6. The method of claim 5, wherein tissue is selected from the group consisting of an embryonic tissue, a fetal tissue, and a post-natal tissue.

7. The method of claim 5, wherein the tissue is an embryonic tissue selected from the group consisting of the aorta-gonad-mesonephros region tissue, yolk sac, and embryonic liver.

8. The method of claim 5, wherein the tissue is a fetal tissue selected from the group consisting of liver, bone marrow, and peripheral blood.

9. The method of claim 5, wherein the tissue is a post-natal tissue selected from the group consisting of cord blood, bone marrow, normal peripheral blood, mobilized peripheral blood, a hepatic tissue, and a splenic tissue.

10. The method of claim 5, wherein the reagent is an antibody.

11. The method of claim 10, wherein the antibody is selected from the group consisting of KDR1 and KDR2.

12. The method of claim 1, wherein the stem cell is isolated using a conjugated vascular endothelial growth factor.

13. The method of claim 1, wherein the differentiated mammalian cell is a human cell.

14. The method of claim 1, wherein the differentiated mammalian cell is a cell of ectodermal origin.

15. The method of claim 1, wherein the differentiated mammalian cell is a cell of mesodermal origin.

16. The method of claim 1, wherein the differentiated mammalian cell is a cell of endodermal origin.

17. The method of claim 1, wherein the differentiated mammalian cell is selected from the group consisting of a skeletal muscle cell, a myocardial cell, an epithelial cell, an endothelial cell, a cartilage cell, a retinal cell, a lens cell, a bone cell, a fat cell, a peripheral nerve cell, a differentiated hematopoietic cell, a marrow stromal cell, a hepatocyte, a splenocyte, a keratinocyte, a fibroblast, a lymphoid cell, and a central nervous system cell.

18. A method of repairing a damaged human tissue, the method comprising
i) maintaining an isolated human KDR⁺ stem cell in the presence of a differentiated mammalian cell of a tissue of the same type as the damaged tissue, whereby the stem cell differentiates to become an altered cell selected from the group consisting of a tissue-exposed stem cell, a precursor of a cell of the same type as the damaged tissue, and a terminally differentiated cell of the same type as the damaged tissue; and
ii) providing the altered cell to the damaged tissue, thereby repairing the tissue.

19. The method of claim 18, wherein the stem cell is maintained in contact with the differentiated mammalian cell.

20. The method of claim 18, wherein the stem cell is separated from the differentiated mammalian cell by a porous barrier.

21. The method of claim 18, wherein the stem cell is isolated from a human hematopoietic tissue using a reagent that specifically binds with KDR.

22. The method of claim 21, wherein tissue is selected from the group consisting of an embryonic tissue, a fetal tissue, and a post-natal tissue.

23. The method of claim 21, wherein the tissue is an embryonic tissue selected from the group consisting of the aorta-gonad-mesonephros region tissue, yolk sac, and embryonic liver.

24. The method of claim 21, wherein the tissue is a fetal tissue selected from the group consisting of liver, bone marrow, and peripheral blood.

25. The method of claim 21, wherein the tissue is a post-natal tissue selected from the group consisting of cord blood, bone marrow, normal peripheral blood, mobilized peripheral blood, a hepatic tissue, and a splenic tissue.

26. The method of claim 21, wherein the reagent is an antibody.

27. The method of claim 26, wherein the antibody is selected from the group consisting of KDR1 and KDR2.

28. The method of claim 21, wherein the stem cell is isolated using a conjugated vascular endothelial growth factor.

29. The method of claim 18, wherein the stem cell is maintained in vitro in the presence of the differentiated mammalian cell.

30. The method of claim 18, wherein the differentiated mammalian cell is a human cell.

31. The method of claim 18, wherein the damaged tissue is associated with a disease or disorder selected from the group consisting of stroke, ischemia, myocardial infarction, coronary artery disease, spinal cord injury, age-related tissue damage, Alzheimer's disease, Parkinson's disease, liver fibrosis, liver cirrhosis, chronic obstructive pulmonary disorder, compartment syndrome, multiple sclerosis, chronic inflammation, chronic infection, macular degeneration, and cataracts.

32. A method of rejuvenating an age-damaged human tissue, the method comprising
i) maintaining an isolated human KDR⁺ stem cell in the presence of a differentiated mammalian cell of a tissue of the same type as the damaged tissue, whereby the stem cell differentiates to become an altered cell selected from the group consisting of a tissue-exposed stem cell, a precursor of a cell of the same type as the damaged tissue, and a terminally differentiated cell of the same type as the damaged tissue; and
ii) providing the altered cell to the age-damaged tissue, thereby rejuvenating the tissue.

33. The method of claim 32, wherein the stem cell is maintained in contact with the differentiated mammalian cell.

34. The method of claim 32, wherein the stem cell is separated from the differentiated mammalian cell by a porous barrier.

35. The method of claim 32, wherein the stem cell is isolated from a human hematopoietic tissue using a reagent that specifically binds with KDR.

36. The method of claim 35, wherein tissue is selected from the group consisting of an embryonic tissue, a fetal tissue, and a post-natal tissue.

37. The method of claim 35, wherein the tissue is an embryonic tissue selected from the group consisting of the aorta-gonad-mesonephros region tissue, yolk sac, and embryonic liver.

38. The method of claim 35, wherein the tissue is a fetal tissue selected from the group consisting of liver, bone marrow, and peripheral blood.

39. The method of claim 35, wherein the tissue is a post-natal tissue selected from the group consisting of cord blood, bone marrow, normal peripheral blood, mobilized peripheral blood, a hepatic tissue, and a splenic tissue.

40. The method of claim 35, wherein the reagent is an antibody.

41. The method of claim 40, wherein the antibody is selected from the group consisting of KDR1 and KDR2.

42. The method of claim 35, wherein the stem cell is isolated using a conjugated vascular endothelial growth factor.

43. The method of claim 32, wherein the stem cell is maintained in vitro in the presence of the differentiated mammalian cell.

44. The method of claim 32, wherein the differentiated mammalian cell is a human cell.

45. The method of claim 32, wherein the differentiated mammalian cell is selected from the group consisting of a skeletal muscle cell, a myocardial cell, an epithelial cell, an endothelial cell, a cartilage cell, a retinal cell, a lens cell, a bone cell, a fat cell, a peripheral nerve cell, a differentiated hematopoietic cell, a marrow stromal cell, a hepatocyte, a splenocyte, a keratinocyte, a fibroblast, a lymphoid cell, and a central nervous system cell.

46. A method of generating a differentiated human cell of a selected type, the method comprising maintaining an isolated human KDR⁺ stem cell in a medium conditioned to reflect the presence of differentiated mammalian cells of the selected type in the medium, whereby the stem cell differentiates to become the differentiated human cell of the selected type.

47. The method of claim 46, wherein the medium is conditioned by culturing differentiated mammalian cells of the selected type therein.

48. The method of claim 47, wherein the differentiated cells are removed from the conditioned medium before maintaining the stem cell in the medium.

49. The method of claim 46, wherein the medium is a synthetic medium made without culturing the differentiated mammalian cells therein.

50. A method of repairing a damaged human tissue, the method comprising
i) maintaining an isolated human KDR⁺ stem cell in a medium conditioned to reflect the presence of differentiated mammalian cells of the same type as the damaged tissue, whereby the stem cell differentiates to become an altered cell selected from the group consisting of a tissue-exposed stem cell, a precursor of a cell of the same type as the damaged tissue, and a terminally differentiated cell of the same type as the damaged tissue; and
ii) providing the altered cell to the damaged tissue, thereby repairing the tissue.

51. The method of claim 50, wherein the medium is conditioned by culturing differentiated mammalian cells of the selected type therein.

52. The method of claim 51, wherein the differentiated cells are removed from the conditioned medium before maintaining the stem cell in the medium.

53. The method of claim 50, wherein the medium is a synthetic medium made without culturing the differentiated mammalian cells therein.

54. A method of rejuvenating an age-damaged human tissue, the method comprising
i) maintaining an isolated human KDR⁺ stem cell in a medium conditioned to reflect the presence of differentiated mammalian cells of the same type as the damaged tissue, whereby the stem cell differentiates to become an altered cell selected from the group consisting of a tissue-exposed stem cell, a precursor of a cell of the same type as the damaged tissue, and a terminally differentiated cell of the same type as the damaged tissue; and
ii) providing the altered cell to the age-damaged tissue, thereby rejuvenating the tissue.

55. The method of claim 54, wherein the medium is conditioned by culturing differentiated mammalian cells of the selected type therein.

56. The method of claim 55, wherein the differentiated cells are removed from the conditioned medium before maintaining the stem cell in the medium.

57. The method of claim 54, wherein the medium is a synthetic medium made without culturing the differentiated mammalian cells therein.

58. An enriched population of long-term repopulating human hematopoietic stem cells obtained using a method comprising obtaining a population of cells from human hematopoietic tissue and isolating a population of KDR⁺ cells therefrom, thereby obtaining a cell population enriched for long-term repopulating human hematopoietic stem cells.

59. A cell obtained using a method comprising obtaining a population of cells from human hematopoietic tissue and isolating a population of KDR⁺ cells therefrom, thereby obtaining a cell population enriched for long-term repopulating human hematopoietic stem cells.

60. The cell of claim 59, wherein said cell comprises an isolated nucleic acid.

61. The cell of claim 60, wherein said isolated nucleic acid is selected from the group consisting of a nucleic acid encoding adenosine deaminase, a nucleic acid encoding beta-globin, a nucleic acid encoding multiple drug resistance, an antisense nucleic acid complementary to a human immunodeficiency virus nucleic acid, an antisense nucleic acid complementary to a nucleic acid encoding a cell cycle gene, and an antisense nucleic acid complementary to a nucleic acid encoding an oncogene.

62. The cell of claim 60, wherein said isolated nucleic acid is operably linked to a promoter/regulatory sequence.

63. The cell of claim 62, wherein said promoter/regulatory sequence is selected from the group consisting of a retroviral long terminal repeat, and the cytomegalovirus immediate early promoter.

64. An isolated purified population of long-term repopulating human hematopoietic stem cells obtained by the method of claim 63.

65. A cell obtained by the method of claim 63.

66. The cell of claim 65, wherein said cell comprises an isolated nucleic acid.

67. The cell of claim 66, wherein said isolated nucleic acid is selected from the group consisting of a nucleic acid encoding adenosine deaminase, a nucleic acid encoding beta-globin, a nucleic acid encoding multiple drug resistance, an antisense nucleic acid complementary to a human immunodeficiency virus nucleic acid, an antisense nucleic acid complementary to a nucleic acid encoding a cell cycle gene, and an antisense nucleic acid complementary to a nucleic acid encoding an oncogene.

68. The cell of claim 66, wherein said isolated nucleic acid is operably linked to a promoter/regulatory sequence.

69. The cell of claim 68, wherein said promoter/regulatory sequence is selected from the group consisting of a retroviral long terminal repeat, and the cytomegalovirus immediate early promoter.

70. A purified population of long-term repopulating human hematopoietic stem cells obtained by a method comprising obtaining a population of cells from human hematopoietic tissue, isolating a population of hematopoietic progenitor cells therefrom, and isolating a population of KDR⁺ cells from said population of hematopoietic progenitor cells, thereby obtaining a purified population of long-term repopulating human hematopoietic stem cells, wherein said hematopoietic progenitor cells are isolated by isolating CD34⁻ using antibody which specifically binds lin to select a population of CD34⁻lin⁻ cells and using an antibody which specifically binds KDR.

71. A cell isolated by a method comprising obtaining a population of cells from human hematopoietic tissue, isolating a population of hematopoietic progenitor cells therefrom, and isolating a population of KDR⁺ cells from said population of hematopoietic progenitor cells, thereby obtaining a purified population of long-term repopulating human hematopoietic stem cells, wherein said hematopoietic progenitor cells are isolated by isolating CD34⁻ using antibody which specifically binds lin to select a population of CD34⁻lin⁻ cells and using an antibody which specifically binds KDR.

72. The cell of claim 71, wherein said cell comprises an isolated nucleic acid.

73. The cell of claim 72, wherein said isolated nucleic acid is selected from the group consisting of a nucleic acid encoding adenosine deaminase, a nucleic acid encoding beta-globin, a nucleic acid encoding multiple drug resistance, an antisense nucleic acid complementary to a human immunodeficiency virus nucleic acid, an antisense nucleic acid complementary to a nucleic acid encoding a cell cycle gene, and an antisense nucleic acid complementary to a nucleic acid encoding an oncogene.

74. The cell of claim 73, wherein said isolated nucleic acid is operably linked to a promoter/regulatory sequence.

75. The cell of claim 74, wherein said promoter/regulatory sequence is selected from the group consisting of a retroviral long terminal repeat, and the cytomegalovirus immediate early promoter.

76. An isolated purified population of long-term repopulating human hematopoietic stem cells obtained by a method comprising obtaining a population of cells from human hematopoietic tissue, isolating a population of KDR⁺ hematopoietic stem cells therefrom, and incubating said population of KDR⁺ cells with vascular endothelial growth factor, thereby expanding said population of long-term repopulating human hematopoietic stem cells.

77. A cell obtained using a method comprising obtaining a population of cells from human hematopoietic tissue, isolating a population of KDR⁺ hematopoietic stem cells therefrom, and incubating said population of KDR⁺ cells with vascular endothelial growth factor, thereby expanding said population of long-term repopulating human hematopoietic stem cells.

78. The cell of claim 77, wherein said cell comprises an isolated nucleic acid.

79. The cell of claim 78, wherein said isolated nucleic acid is selected from the group consisting of a nucleic acid encoding adenosine deaminase, a nucleic acid encoding beta-globin, a nucleic acid encoding multiple drug resistance, an antisense nucleic acid complementary to a human immunodeficiency virus nucleic acid, an antisense nucleic acid complementary to a nucleic acid encoding a cell cycle gene, and an antisense nucleic acid complementary to a nucleic acid encoding an oncogene.

80. The cell of claim 79, wherein said isolated nucleic acid is operably linked to a promoter/regulatory sequence.

81. The cell of claim 80, wherein said promoter/regulatory sequence is selected from the group consisting of a retroviral long terminal repeat, and the cytomegalovirus immediate early promoter.

82. A blood substitute comprising the progeny cells of an isolated purified population of long term repopulating human hematopoietic stem cells.

83. The blood substitute of claim 82, wherein said progeny cells are selected from the group consisting of red blood cells, neutrophilic granulocytes, eosinophilic granulocytes, basophilic granulocytes, monocytes, dendritic cells, platelets, B lymphocytes, T lymphocytes, natural killer cells, and differentiated precursors thereof, and undifferentiated progenitors thereof.

84. A chimeric non-human mammal comprising at least one of an isolated and purified long-term repopulating human hematopoietic stem cell.

85. The chimeric mammal of claim 84, wherein said cell is introduced into said mammal using a method selected from the group consisting of transplantation, and blastocyst injection.

86. The non-human mammal of claim 85, wherein said mammal is selected from the group consisting of a mouse, a rat, a dog, a donkey, a sheep, a pig, a horse, a cow, a non-human primate.

87. A method of inhibiting rejection of a transplanted organ, said method comprising ablating the bone marrow of a transplant recipient and administering to said recipient a multi-lineage engrafling dose of an isolated and purified long-term repopulating human hematopoietic stem cell obtained from the hematopoietic tissue of the donor of said organ, thereby inhibiting rejection of a transplanted organ.

88. A method of transplanting an autologous human hematopoietic stem cell in a human, said method comprising obtaining a population of cells from the hematopoietic tissue of a human and isolating a population of non-malignant hematopoietic stem cells therefrom, ablating the bone marrow of said human, and administering at least one said isolated non-malignant hematopoietic stem cell to said human, thereby transplanting an autologous human hematopoietic stem cell in a human.

89. A method of monitoring the presence of KDR⁺ stem cells in a human hematopoietic tissue in a human receiving therapy, said method comprising obtaining a sample of hematopoietic tissue from said human before, during and after said therapy, and measuring the number of KDR⁺ stem cells in said sample, thereby monitoring the presence of KDR⁺ stem cells in a human hematopoietic tissue obtained from a human receiving therapy.


SEQUENCE LISTING (10 sequences)

SEQ ID NO: 1 - DNA, 22 bp, Artificial Sequence
  Description of Artificial Sequence PCR Primer
aaaaccttttgttgcttttgga

SEQ ID NO: 2 - DNA, 22 bp, Artificial Sequence
  Description of Artificial Sequence PCR Primer
gaaatgggattggtaaggatga

SEQ ID NO: 3 - DNA, 22 bp, Artificial Sequence
  Description of Artificial Sequence PCR Primer
aaaccaagactagatagcgtca

SEQ ID NO: 4 - DNA, 22 bp, Artificial Sequence
  Description of Artificial Sequence PCR Primer
ttctcacataatcggggttctt

SEQ ID NO: 5 - DNA, 22 bp, Artificial Sequence
  Description of Artificial Sequence PCR Primer
gacaaggagtgtgaccactgaa

SEQ ID NO: 6 - DNA, 22 bp, Artificial Sequence
  Description of Artificial Sequence PCR Primer
tgaagggacattgtgtgagaag

SEQ ID NO: 7 - DNA, 24 bp, Artificial Sequence
  Description of Artificial Sequence PCR Primer
gagtccttctttgggagatagtga

SEQ ID NO: 8 - DNA, 24 bp, Artificial Sequence
  Description of Artificial Sequence PCR Primer
gtcagactggtcacaggttagaca

SEQ ID NO: 9 - DNA, 24 bp, Artificial Sequence
  Description of Artificial Sequence PCR Primer
catttttgcagagaacaacatagg

SEQ ID NO: 10 - DNA, 24 bp, Artificial Sequence
  Description of Artificial Sequence PCR Primer
tcaagcactggataaattgtagga